(19) United States
(12) Patent Application Publication
(10) Pub. No.: US 2018/0105832 A1
(43) Pub. Date: Apr. 19, 2018

(54) NOVEL AFLATOXIN AND FUNGAL INFECTION CONTROL METHODS

(71) Applicant: Fraunhofer-Gesellschaft zur Foerderung der angewandten Forschung e.V., Munich (DE)

(72) Inventors: Greta Noelke, Aachen (DE); Stefan Schillberg, Aachen (DE); Max Schubert, Aachen (DE)

(21) Appl. No.: 15/566,446

(22) PCT Filed: Apr. 13, 2015
(86) PCT No.: PCT/EP2015/057943

§ 371 Date: Oct. 13, 2017

(51) Int. Cl. C12N 15/82; C07K 16/14; C12N 15/113; A61P 31/10; A61P 1/16; A61P 35/00; A61P 25/00; A61P 25/08; A61P 7/10; A61P 7/04; A61P 1/00; A61P 3/00

(52) U.S. Cl. CPC C12N 15/8282; C07K 16/14; C12N 15/113; A61P 31/10; A61P 1/16; A61P 35/00; C12N 2310/14; A61P 25/08; A61P 7/10; A61P 7/04; A61P 1/00; A61P 3/00; C07K 2317/34; A61P 25/00

(57) ABSTRACT

The technology provided herein relates to novel methods and compounds for a multi-species pathogen infection control. In particular, the present disclosure pertains to methods of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP. Nucleic acid molecules encoding said inhibitors, vectors and host cells containing the nucleic acids and methods for preparation and producing such inhibitors are also disclosed, as well as the use of said CSP-inhibitors for the control/treatment of diseases associated with a microbial pathogen expressing CSP.

### FIELD OF THE DISCLOSURE

The technology provided herein relates to novel methods and compounds for a multi-species pathogen infection control. In particular, the present disclosure pertains to methods of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP. Nucleic acid molecules encoding said inhibitors, vectors and host cells containing the nucleic acids and methods for preparation and producing such inhibitors are also disclosed, as well as the use of said CSP-inhibitors for the control/treatment of diseases associated with a microbial pathogen expressing CSP.

### BACKGROUND

Aflatoxins produced as secondary metabolites by soil-borne molds are the most toxic, naturally occurring carcinogens known in the fungal kingdom. The main aflatoxin producers, Aspergillus flavus and A. parasiticus, are ubiquitous in nature, have no specificity towards their hosts and therefore can infect a large number of different seeds of cereals, nut beans, coffee beans and oil-rich seeds during cultivation, harvest and post-harvest storage, generating high levels of aflatoxins especially under humid storage conditions.

Aflatoxins are stable during food processing and can be enriched in the food chain. Aflatoxin contaminated diet has been directly linked with the elevated rate of liver cancer, decreased immunity, kwashiorkor and growth stunting. Outbreaks of aflatoxicosis are common in tropical countries, mostly among adults in poorly nourished rural populations whose staple food is maize.

Aflatoxin producing fungal species has no specificity towards their hosts and therefore can infect a large number of different seeds of cereals, nut beans, coffee beans and oil-rich seeds before and after harvest as well as during the storage. Worldwide, Aspergillus species cause significant losses in major crops reaching up to more than 20%. The annual economic impact of aflatoxin contamination on corn and peanut agriculture in the USA is thought to exceed USD 1 billion (Agricultural research, 2013).

The production of aflatoxin-free crops is challenging because there are no effective methods to prevent aflatoxin production. Current disease and post-harvest control measures are expensive and especially chemical fungicides remain a major input in the costs of crop production in many parts of the world.

More recent molecular breeding strategies allow the targeted introduction of resistance traits into crops and have been used to develop aflatoxin-resistant maize lines. However, the identification of resistant germplasm from exotic maize lines and their introgression into commercial lines requires breeding over many generations. However, aflatoxin production in the field is highly influenced by environmental changes and maize lines classified as resistant to Aspergillus and aflatoxin exhibit undesired traits in respect to a commercialization of high-performing crop lines (Brown et al. 2013). These challenges are time-consuming and have driven the development of alternative strategies based on genetic engineering to control the Aspergillus fungi responsible for aflatoxin production. There are currently no commercially viable cultivars that suppress aflatoxin accumulation in the field.

To reduce the amount of fungicides, new biotechnical approaches are developed. One of these approaches is the use of RNA interference. With regard to Aspergillus, RNAi-mediated gene silencing was achieved in A. flavus to silence in particular key genes of aflatoxin pathway (Abdel-Hadi et al. 2010; Abdel-Hadi et al. 2011; McDonald et al. 2005). Furthermore silencing of key genes stcJ, stcK and stcA of sterigmatocystin, which catalyze rate-limiting steps of pre-building blocks in aflatoxin was demonstrated as an efficient mean to reduce and control aflatoxin production in Aspergillus (Alakonya and Monda 2013; Yu and Ehrlich 2011).

However, the availability of novel and improved methods and compounds to control target pathogens, in particular aflatoxin-producing pathogens would be highly advantageous.

### SUMMARY OF THE DISCLOSURE

The present disclosure pertains to novel methods and compounds for a multi-species pathogen control, in particular for aflatoxin and fungal infection control. The present disclosure pertains to methods of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP. Nucleic acid molecules encoding said inhibitors, vectors and host cells containing the nucleic acids and methods for preparation and producing such inhibitors are also disclosed, as well as the use of said CSP-inhibitors for the use of the control and/or treatment of aflatoxin-producing fungi in agriculture and/or for the treatment of a disease associated with a pathogen expressing CSP.

Therefore, in a first aspect, the present disclosure relates to methods of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP.

In a second aspect, embodiments of this disclosure relate to isolated polynucleotides selected from the group consisting of:
a) a polynucleotide derived from a nucleic acid sequence selected from the group consisting of SEQ ID NO:1 or SEQ ID NO:2.
b) a polynucleotide comprising a nucleic acid sequence selected from the group consisting of SEQ ID NO:1 or SEQ ID NO:2.
c) a polynucleotide that hybridizes to a nucleic acid sequence selected from the group consisting of SEQ ID NO:1 or SEQ ID NO:2 under stringent conditions;
d) a polynucleotide of at least 70, at least 80, at least 85, at least 90 percent sequence identity, to a nucleic acid sequence selected from the group consisting of SEQ ID NO:1 or SEQ ID NO:2;
e) a fragment of at least 16 contiguous nucleotides of a nucleic acid sequence selected from the group consisting of SEQ ID NO:1 or SEQ ID NO:2; and
f) a complement of the sequence of (a), (b), (c), (d) or (e).

A third aspect pertains to plants transformed, transduced or transfected with an isolated polynucleotide according to the present disclosure.

In a fourth aspect, the present disclosure relates to methods for controlling an aflatoxin-production fungal pathogen infection comprising providing an agent comprising a first polynucleotide sequence that functions upon uptake by the fungi to inhibit a biological function within said fungi, wherein said polynucleotide sequence exhibits from about 95 to about 100 percent nucleotide sequence identity along at least from about 16 to about 30 contiguous nucleotides to a CSP coding sequence derived from said fungi and is hybridized to a second polynucleotide sequence that is complementary to said first polynucleotide sequence.

In a fifth aspect, the present disclosure pertains to transgenic plants comprising a gene coding an inhibitor against CSP of a target pathogen, wherein said inhibitor inhibits the CSP expression and/or binds to a protein product of a gene coding CSP.

In a sixth aspect, some embodiments of this disclosure relate to plants transformed with a polynucleotide according to the present disclosure, or a seed thereof comprising said polynucleotide.

Further, some embodiments pertain to commodity products produced from a plant according to the fifth aspect, wherein said commodity product comprises a detectable amount of a polynucleotide according to the second aspect or a ribonucleotide expressed therefrom.

In a further aspect, some embodiments provide methods for controlling aflatoxin contamination and plant infection with Aspergillus comprising providing inside Aspergillus fungi an agent comprising a first polynucleotide sequence that functions upon uptake by the fungi to inhibit a biological function within said fungi, wherein said polynucleotide sequence exhibits from about 58 to about 100 percent nucleotide sequence identity along at least from about 16 to about 25 contiguous nucleotides to a CSP coding sequence derived from said Aspergillus pathogen and is hybridized to a second polynucleotide sequence that is complementary to said first polynucleotide sequence, and wherein said coding sequence derived from said pathogen comprise a sequence selected from the group consisting of SEQ ID NO:1 and SEQ ID NO. 2, or a complement thereof.

Further, in a seventh aspect, embodiments of the present disclosure pertains to methods for controlling a CSP expressing Aspergillus pathogen, wherein a plant cell expressing a polynucleotide sequence according to the present disclosure, wherein the polynucleotide is expressed to produce a double stranded ribonucleic acid, wherein said double stranded ribonucleotide acid and/or a RNAi inducing compound derived from said double stranded ribonucleotide acid functions upon uptake by the fungi to inhibit the expression of a CSP encoding target sequence within said fungi and results in decreased fungal growth and plant cell infection.

Further, in an eight aspect, embodiments of the present disclosure pertains to method for improving the yield of a crop produced from a crop plant subjected to a CSP-expressing pathogen infection like an Aspergillus pathogen infection, said method comprising the steps of,
a) introducing an isolated polynucleotide according to the present disclosure into said crop plant,
b) cultivating the crop plant to allow the expression of said polynucleotide, wherein expression of the polynucleotide inhibits growth and infection of fungal pathogen and loss of yield due to pathogen infection.

In a further aspect, the present disclosure relates to transgenic plant comprising a gene coding an inhibitor against CSP of a target fungal pathogen.

In a further aspect, the present disclosure relates also to methods of treating a disease associated with a pathogen as defined in the present disclosure comprising administering an effective amount of an inhibitor as defined in the present disclosure to a patient in need thereof.

Before the disclosure is described in detail, it is to be understood that this disclosure is not limited to the particular component parts of the process steps of the methods described. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1 shows the reactivity of CSP-specific mAbAP10 towards A. flavus and A. parasiticus cell wall fragments as determined by ELISA.

FIG. 2 shows the indirect binding of CSP-specific mAbAP10 to freshly-harvested A. flavus conidia (A) and mycelia germinated overnight (B) visualized by immunofluorescence microscopy. The specific binding of mAbAP10 to the spore surface but not to the germinated mycelia indicates the localization of CSP on the fungal surface only at the early stages of development. Scale bars=50 μm.

FIG. 3 presents an overview of an assay to identify CSP-expressing target pathogens.

FIG. 4 shows the amino acid sequence (A) of CSP (SEQ ID NO. 3) and (B) the epitope recognized by mAbAP10. The signal peptide is shown in italic and underlined, and peptide sequences found in protein spots 1 and 2 are shown in bold. The sequence homologies found in spots 1 and 2 are highlighted in grey.

FIG. 5 shows the dose-dependent effect of CSP silencing using dsRNA derived from SEQ ID NO.1 (namely SEQ ID NO. 2) on the growth of A. flavus, compared to a water-only control. Serial dilutions (from 0.025 to 4 nM) of CSP-specific siRNA (or the water-only control) were incubated with 200 A. flavus conidia for 12 h at 28° C. in the dark. Mycelia stained with calcofluor white were visualized using the Opera® High Content Screening confocal microscope. Scale bars=100 μm.

FIG. 6 shows the quantitative growth inhibition achieved by silencing with CSP-specific siRNA (SEQ ID NO. 2) in A. flavus (A) and A. parasiticus (B). The reduction of fungal growth following incubation with CSP-specific siRNA was statistically significant.

FIG. 7 shows a cDNA containing the part of the mRNA sequence encoding A. flavus CSP (SEQ ID NO.1).

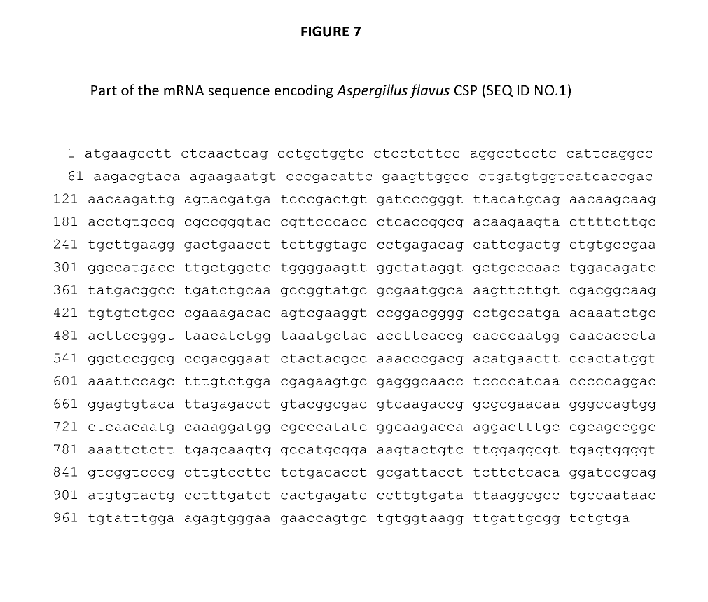

FIG. 8 shows (A) the cDNA nucleic acid sequence of a siRNA (SEQ ID NO.2) derived from SEQ ID NO.1, (B) the amino acid sequence of the A. flavus CSP (SEQ ID NO: 3), (C) the amino acid sequence of the mAbAP10 heavy chain variable regions (SEQ ID NO: 4), and (D) the amino acid sequence of the mAbAP10 light chain variable regions (SEQ ID NO: 5).

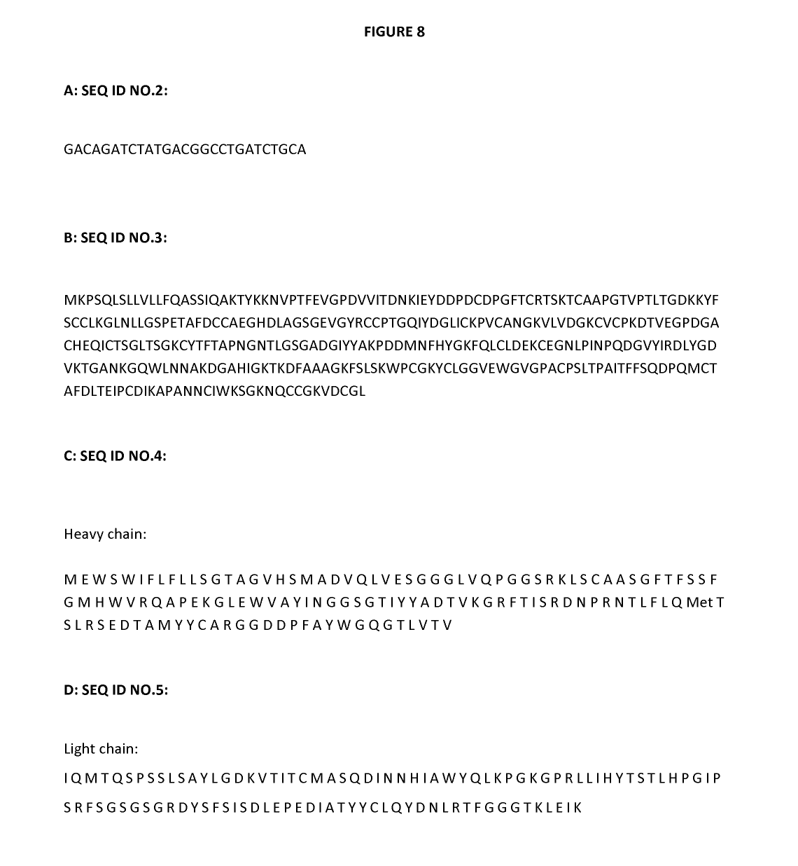

Table 1 summarizes the cross reactivity of Aspergillus-specific mAbAP10 against the cell wall proteins of several fungal pathogens, as determined by ELISA.

### DETAILED DESCRIPTION OF THE DISCLOSURE

Disclosed herein are novel methods and compounds for a multi-species CSP-expressing pathogen control like an aflatoxin and fungal infection control. In particular, the present disclosure pertains to methods of inhibiting the growth of a target pathogen expressing the cysteine-rich secreted protein (CSP), whereby said methods comprise contacting said target pathogen with an inhibitor against said CSP, wherein said inhibitor inhibits the CSP expression and/or binds to a protein product of a gene coding CSP. Nucleic acid molecules encoding said inhibitors, vectors and host cells containing the nucleic acids and methods for preparation and producing such inhibitors are also disclosed, as well as the use of said CSP-inhibitors for the control and/or treatment of aflatoxin-producing fungi in agriculture and/or for the treatment of a disease associated with a pathogen expressing CSP. In particular, the present disclosure provides methods and compositions for genetic control of an aflatoxin-producing Aspergillus strain infection.

The inhibitors and growth inhibition methods of the present disclosure are markedly useful, since they can significantly inhibit growth of undesirable pathogens, in particular of aflatoxin-producing fungi, in particular in agriculture.

Embodiments of the present disclosure pertains to novel aflatoxin and fungal infection control methods comprise the incorporation of an inhibitor against the cysteine-rich secreted protein (CSP) inside to the body of an agricultural and/or human and/or animal target pathogen, in particular against fungal pathogens of the phylum Ascomycota, in particular against the fungi Aspergillus flavus and/or Aspergillus parasiticus, and to pathogen control agents to be used in the method and to transgenic crop, greenhouse and ornamental plants.

Surprisingly, the inventors found that inhibiting CSP is a universally applicable form of a multi-species pathogen infection control like for a control of fungi belonging to the genera of Aspergillus like Aspergillus flavus, Aspergillus parasiticus and Aspergillus fumigatus. In particular, the inventors identify the CSP-encoding genes as target genes in several pathogens, which are for example suitable for reverse genetics of RNAi-mediated gene silencing. For example, the generation of transgenic plants expressing dsRNA targeting the CSP encoding nucleic acid in fungal pathogens could be an efficient and environmentally sustainable approach to reduce the impact of fungal infection and aflatoxin contamination on agriculture. The inventor further identified that that silencing of CSP in the fungi, for example induced by application of specific double stranded RNA, prevents fungal growth. This could lead to later and/or reduced and/or no infection in plants susceptible to the Aspergillus infection in comparison to control groups. It can be assumed that CSP-inhibition in aflatoxin-producing pathogens like fungi belonging to the genera of Aspergillus will decrease the aflatoxin contamination in agricultural products and food chain.

As used herein, the phrase “encoding nucleic acid”, “coding sequence”, “encoding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

The term “gene” refers to a DNA sequence that comprises control and coding sequences necessary for the production of a recoverable bioactive polypeptide or precursor.

The term “complementary” as used herein refers to a relationship between two nucleic acid sequences. One nucleic acid sequence is complementary to a second nucleic acid sequence if it is capable of forming a duplex with the second nucleic acid, wherein each residue of the duplex forms a guanosine-cytidine (G—C) or adenosine-thymidine (A—T) base pair or an equivalent base pair. Equivalent base pairs can include nucleoside or nucleotide analogues other than guanosine, cytidine, adenosine, or thymidine.

Advantageous embodiments of the present disclosure pertains to methods of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP.

The pathogen growth inhibitory effect of the method and inhibitor of the present disclosure can be confirmed by the method which is described later in the examples.

According to the present disclosure, the target pathogen is a pathogen expressing the cysteine-rich secreted protein (CSP). Gene expression is the process by which information from a gene is used in the synthesis of a functional gene product, namely the cysteine-rich secreted protein (CSP).

As used herein, the phrase “inhibition of gene expression” or “inhibits the CSP expression” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule.

CSP (gi 238486514) is described in the literature as a hypothetical protein with unknown function (Payne et al. 2006). The inventor identified with the present disclosure the first time that silencing of CSP in a target pathogen (an Aspergillus strain) induced by incubation of the pathogen with specific double stranded RNA, prevents cell growth. The results according to the present disclosure demonstrate that the inhibition of pathogen cell growth strongly correlated with the amounts of siRNAs. The complete reduction of fungal growth after silencing of CSP with CSP-specific siRNAs suggests that CSP has a crucial role in the life cycle of a CSP-expressing pathogen. It can be assumed that inhibition of e.g. fungal growth will decrease the levels of aflatoxines produced from this fungi and will lower their negative influences of food contamination in the human and livestock. The term “variant of CSP” refers herein to a polypeptide which is substantially similar in structure and biological activity to a CSP according to one of the disclosed sequences or encoded by one of the disclosed sequences.

In some advantageous embodiments, the CSP is coded by an mRNA comprising SEQ ID NO: 1, or homologs thereof encoding a functional CSP in the target pathogen. In some embodiments, the expressed CSP comprises the amino acid sequence of SEQ ID NO.3.

In general, the term “homolog” or “homologue” according to the present disclosure includes amino acid or nucleic acid sequences having a sequence identity of at least 50%, in particular of at least 60%, particular of at least 70%, in particular of at least 85%, in particular of at least 85%, in particular of at least 90%, in particular of at least 95%, in particular of at least 96, 97, 98 or 99% to the parent sequence. The term “homologue” or “homologue” in view of a nucleic acid molecule refers also to a nucleic acid molecule, wherein the sequence has one or more nucleotides added, deleted, substituted or otherwise chemically modified in comparison to a parent nucleic acid molecule according to one of the disclosed sequences, provided always that the homologue encoding a functional CSP in the target pathogen. The term “homologue of the nucleic acid molecule” refers to a nucleic acid molecule which has one or more nucleotides added, deleted, substituted or otherwise chemically modified in comparison to a nucleic acid molecule according to one of the sequences disclosed herein, provided always that for the inhibitory nucleic acids described herein the homologue nucleic acids retains substantially the same inhibitory effect on CSP expression.

As used herein, the term “homologous” or “homologs”, with reference to a nucleic acid sequence, includes a nucleotide sequence that hybridizes under stringent conditions to one of the coding sequences of SEQ ID NO: 1 and/or SEQ ID NO: 2, or the complements thereof. Sequences that hybridize for example under stringent conditions to SEQ ID NO: 1 and/or SEQ ID NO: 2, or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. Substantially homologous sequences have preferably from about 70% to about 80% sequence identity, or more preferably from about 80% to about 85% sequence identity, or most preferable from about 90% to about 95% sequence identity, to about 99% sequence identity, to the referent nucleotide sequences of SEQ ID NO: 1, or to the sequence of SEQ ID NO: 2 as set forth in the sequence listing, or the complements thereof.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

Therefore, in some advantageous embodiments, the CSP is coded by a mRNA comprising SEQ ID NO: 1, or homologs thereof, wherein said homologs have a sequence identity of at least 60%, in particular of at least 70%, in particular of at least 85%, in particular of at least 85%, in particular of at least 90%, in particular of at least 95%, in particular of at least 96, 97, 98 or 99% to SEQ ID NO: 1 or parts thereof and encodes a functional CSP in the target pathogen.

As mentioned above, “Percent sequence identity”, with respect to two amino acid or polynucleotide sequences, refers to the percentage of residues that are identical in the two sequences when the sequences are optimally aligned. Thus, 80% amino acid sequence identity means that 80% of the amino acids in two optimally aligned polypeptide or polynucleic sequences are identical. Percent identity can be determined, for example, by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis.

In advantageous embodiments of the present disclosure, the target pathogen is a CSP-expressing microorganism, in particular a CSP-expressing fungus like an Aspergillus flavus or Aspergillus parasiticus. The term “fungus” is not particularly limited as long as it belongs to fungi and the fungi are expressing the CSP. In further advantageous embodiments, the target pathogen is an aflatoxin-producing fungus. As mentioned above, Aflatoxins are naturally occurring mycotoxins that are produced for example by Aspergillus flavus and Aspergillus parasiticus. Aflatoxin-producing fungi include fungi producing at least one type of aflatoxin like Aflatoxin B1, Aflatoxin B2, Aflatoxin G1, Aflatoxin G2, Aflatoxin M1, Aflatoxin M2, Aflatoxicol, and Aflatoxin Q1 (AFQ1).

In some embodiments, the target pathogen is a fungus belonging to the phylum Ascomycota, in particular a fungus belonging to the order Eurotiales, in particular a fungus belonging to the family Trichocomaceae and in particular a fungus belonging to the genera Aspergillus.

In particular, many Aspergillus fungi are important agricultural pathogens because they cause direct damage of the agronomical important crops and produce aflatoxins which cause damage to their host plants and cause also serious diseases in humans and/or animals.

Therefore, in advantageous embodiments the target pathogen is a CSP-expressing fungus belonging to the genera of Aspergillus, in particular Aspergillus flavus and/or Aspergillus parasiticus. In a further embodiment, the target pathogen is Aspergillus fumigatus. Furthermore, the target pathogen is Aspergillus fischerianus.

For example, a target pathogen expressing CSP can be identified by using an antibody or an antibody fragment against CSP like the monoclonal antibody mAbAP10 as described in the present disclosure. SEQ ID No. 4 and SEQ ID No. 5 show the amino acid sequence of the light chain and heavy chain of the variable regions in the monoclonal antibody mAbAP10. An assay to identify CSP on a target pathogen is shown in example 3 of the present disclosure. Further methods for the identification of a target pathogen expressing CSP could be an ELISA as known in the prior art.

The present disclosure pertains to aflatoxin and fungal control methods comprising incorporating an inhibitor against the cysteine-rich secreted protein (CSP) inside an agricultural and/or human and/or animal target pathogens expressing CSP. In particular, the mRNA encoding the CSP comprises the sequence set forth in SEQ ID NO: 1, or homologs thereof, wherein said homologs may have a sequence identity of at least 80%, in particular of at least 85%, in particular of at least 90% to SEQ ID NO: 1. In an advantageous example said homologs are parts of sequences that encode a functional CSP in the target pathogen.

In the present description, “fungal control” refers to the removal or the reduction of harm of pathogen. The concept of “fungal control” include reducing the growth of the target pathogen, killing of pathogen (extermination), pathogen proliferation inhibition, pathogen growth inhibition, repelling of pathogen (repellence), and the removal or the reduction of harm of pathogen (for example, inhibition of aflatoxin production

In some advantageous embodiments of the present disclosure, the inhibitor for inhibiting the growth of a target pathogen expressing the cysteine-rich secreted protein (CSP) is a compound selected from the group consisting of the following (a) to (f):
(a) an RNAi inducing compound targeted a nucleic acid coding CSP or parts thereof;
(b) a nucleic acid construct intracellularly producing an RNAi inducing compound targeted a nucleic acid coding CSP or parts thereof;
(c) an antisense nucleic acid targeted at the transcript product of a gene coding CSP or parts thereof;
(d) a ribozyme targeted at the transcript product of a gene coding CSP or parts thereof;
(e) a small chemical molecule targeted the protein product of a gene coding CSP;
(f) a peptide or polypeptide targeted the protein product of a gene coding CSP.

The term “Inhibitor” or “CSP inhibitor” is used as the generic name of the substances inhibiting CSP, in particular said inhibitor inhibits the CSP expression and/or binds to a protein/polypeptide product of a gene coding CSP. For example, the CSP inhibitor may inhibit the expression, the transcription and/or the translation of CSP and/or has inhibitory activity against the expressed CSP.

Therefore, the present disclosure provides recombinant DNA technologies to post-transcriptionally repress or inhibit expression of a target cysteine-rich secreted protein (CSP) coding sequence in the cell of a target pathogen like a pathogenic fungi to provide a pathogen-protective effect by uptake of one or more double stranded RNA (dsRNA) and/or small interfering ribonucleic acid (siRNA) molecules transcribed from all or a portion of a target coding sequence, thereby controlling the pathogen infection. Therefore, the present disclosure relates in particular to sequence-specific inhibition of expression of CSP-coding sequences using double-stranded RNA (dsRNA), including small interfering RNA (siRNA), to achieve the intended levels of fungi control.

The term “isolated” describes any molecule separated from its natural source.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

Provided according to the disclosure are nucleotide sequences, the expression of which results in an RNA sequence which is substantially homologous to an RNA molecule of a targeted gene encoding CSP in an target pathogen like a fungus that comprises an RNA sequence encoded by a nucleotide sequence within the genome of the target pathogen, in particular of the fungus. Thus, after uptake of the stabilized RNA sequence down-regulation of the nucleotide sequence of the target gene in the cells of the target pathogen, in particular of the fungi may be obtained resulting in a deleterious effect on the maintenance, viability, proliferation, reproduction and infestation of the target pathogen like fungi.

Isolated and substantially purified nucleic acid molecules including but not limited to non-naturally occurring nucleotide sequences and recombinant DNA constructs for transcribing dsRNA molecules of the present disclosure are provided that suppress or inhibit the expression of target coding sequence for the cysteine-rich secreted protein (CSP) in the target pathogen like a pathogen fungus when introduced thereto.

Inhibitors according to (a) and (b) are compounds used for the inhibition of expression by so-called RNAi (RNA interference). In other words, when the compound (a) or (b) is used, the expression of CSP is inhibited by RNAi, whereby pathogen control effect is achieved. In this manner, the use of RNAi allows specific control of the target pathogen, and facilitates rapid achievement of pathogen control effect. Furthermore, owing to its properties, the possibility of occurrence of resistant strains is likely extremely low. In addition, RNAi does not modify plant genes, and thus will not genetically influence them.

The “RNAi” refers to the inhibition of expression of the target gene by the introduction of an RNA composed of a sequence homologous to that of the target gene (specifically homologue to the mRNA corresponding to the target gene) into the target cell. For the inhibition of expression using the RNAi method in target pathogens such as fungi, generally, a dsRNA (double strand RNA) composed of a sequence corresponding a part of the target gene (the gene coding the IAP of the target pathogen) like a sequence corresponding SEQ ID No. 1 or a sequence derived from SEQ ID No. 1, for example SEQ ID. NO. 2. Two or more dsRNAs may be used for one target gene.

As used herein, the term “derived from” refers to a specified nucleotide sequence that may be obtained from a particular nucleic acid sequence. As used herein the term “nucleic acid sequence derived from” or “nucleotide acid sequence derived from” refers to polynucleotides comprising the nucleic acid sequence of the full-length parent polynucleotide or in particular only parts of the polynucleotides, like shorter nucleic acid sequence. Therefore, the term refers in particular to a continuous part of the full-length nucleic acid sequence like SEQ ID NO. 1 with or without mutations, which is separate from and not in the context of the full-length nucleic acid sequence. The term “derived from” include short nucleic acids like DNA or RNA derived from SEQ ID NO.1 (like SEQ ID NO. 2), or from homologs thereof, wherein said homologs may be at least 85% identical to the nucleic acid sequence of SEQ ID NO. 1. In particular, nucleic acid sequence derived from SEQ ID NO. 1, or homologs thereof having at least 60%, in particular of at least 70%, in particular of at least 85%, in particular of at least 85%, in particular of at least 90%, in particular of at least 95%, in particular of at least 96, 97, 98 or 99% to SEQ ID NO: 1.identity to SEQ ID NO. 1.

The RNAi targeted at the gene of a mammal cell uses a short dsRNA (siRNA) of about 15 to 30 nucleotides, in particular 27 nucleotides. The use of a dsRNA is preferred for inducing effective inhibition of expression, but the use of a single strand RNA will also be contemplated. The dsRNA used herein is not necessarily composed of two molecules of sense and antisense strands, and, for example, may have a structure wherein the sense and antisense strands composing the dsRNA are connected via a hairpin loop. A dsRNA composed of a modified RNA may be used. Examples of the modification include phosphorothioation, and the use of a modified base (for example, fluorescence-labeled base). In advantageous embodiments, the RNAi inducing compound is a compound selected from the group consisting of short interfering nucleic acids (siNA), short interfering RNA (siRNA), microRNA (miRNA), short hairpin RNAs (shRNA) and precursors thereof which are processed in the cell to the actual RNAi inducing compound. In a preferred embodiment, the precursor is double-stranded RNA (dsRNA). An example of a dsRNA used in the pathogen control method according to the present disclosure is a dsRNA comprising the sequence set forth in SEQ ID NO: 1, or homologs thereof. Furthermore, a dsRNA used in the pathogen control method according to the present disclosure is a dsRNA comprising the sequence set forth in SEQ ID NO: 2, or homologs thereof. An RNAi specific to the target gene can be also produced by intracellularly expression of a dsRNA targeted at the target gene. The nucleic acid construct (b) is used as such a means.

The dsRNA used in the RNAi method may be prepared by chemical synthesis, or in vitro or in vivo using an appropriate expression vector. The method using an expression vector is particularly effective for the preparation of a relatively long dsRNA. The design of dsRNA normally includes the sequence (continuous sequence) specific to the target nucleic acid. Programs and algorithms for selecting an appropriate target sequence have been developed.

Embodiments of the present disclosure pertains also to small interfering ribonucleic acids (siRNAs) for inhibiting the expression of a the cysteine-rich secreted protein (CSP) protein in a target pathogen, wherein the siRNA comprises at least 2 sequences that are complementary to each other and wherein a sense strand comprises a first sequence and an anti-sense strand comprises a second sequence comprising a region of complementarity, which is substantially complementary to at least a part of an mRNA encoding a nucleotide sequence from SEQ ID NO. 1. In an advantageous embodiment, the siRNA contains the sequence of SEQ ID. NO.2, or homologs thereof, wherein said homologs have a sequence identity of at least 60%, in particular of at least 70%, in particular of at least 85%, in particular of at least 85%, in particular of at least 90%, in particular of at least 95%, in particular of at least 96, 97, 98 or 99% to SEQ ID NO: 2.

Therefore, the present disclosure pertains to methods for controlling aflatoxin-production and/or fungal infection comprising providing an agent comprising a first polynucleotide sequence that functions upon uptake by the fungi to inhibit a biological function within said fungi, wherein said polynucleotide sequence exhibits from about 95 to about 100 percent nucleotide sequence identity along at least from about 15 to about 30 contiguous nucleotides, in particular from about 16 to 27 contiguous nucleotides, to a CSP coding sequence derived from said fungi and is hybridized to a second polynucleotide sequence that is complementary to said first polynucleotide sequence. In some advantageous embodiments, said CSP coding sequence derived from said fungi is selected from the group consisting of SEQ ID NO:1 and SEQ ID NO:2, or complements thereof.

As mentioned above, advantageous embodiments of the present disclosure pertain to the use of RNA interference to silence the expression of CSP in a target pathogen to disrupt growth of the target pathogen like an aflatoxin-producing fungi and therefore plant infection and aflatoxin production were reduced.

The above described (c) is a compound used for the inhibition of expression by an antisense method. The inhibition of expression using an antisense method is generally carried out using an antisense construct that produces an RNA complementary to the portion specific to the mRNA corresponding to the target gene upon transcription. The antisense construct (also referred to as antisense nucleic acid) is, for example, introduced into the target cell in the form of an expression plasmid. The antisense construct may be an oligonucleotide probe that hybridizes with the DNA sequence or corresponding mRNA sequence of the target gene (these sequences may be collectively referred to as “target nucleic acid”) upon introduction into the target cell, and inhibits their expression. The oligonucleotide probe is preferably resistant to endogenous nucleases such as exonuclease and/or endonuclease. When a DNA molecule is used as an antisense nucleic acid, the DNA molecule is preferably an oligodeoxyribonucleotide derived from the region containing the translation initiation site of the mRNA corresponding to the target gene (for example, the region from −10 to +10). In some advantageous embodiments, target nucleic acid comprises a nucleic acid having SEQ ID NO. 1, or homologs thereof. Therefore, the antisense nucleic acid as a CSP-inhibitor hybridize to SEQ ID NO 1, or homologs thereof.

The complementation between the antisense nucleic acid and target nucleic acid is preferably precise, but some mismatch may occur. The hybridization capacity of the antisense nucleic acid for the target nucleic acid generally depends on the degree of complementation between the nucleic acids and the length of the antisense nucleic acid. In principle, the longer the antisense nucleic acid, the more stable double strand (or triplex) is formed between the antisense and target nucleic acids, even if many mismatches occur. Those skilled in the art can examine the degree of acceptable mismatch using a standard method.

The antisense nucleic acid may be DNA, RNA, or a chimera mixture thereof, or a derivative or modified product thereof. The antisense nucleic acid may be single or double strand. The stability and hybridization capacity of the antisense nucleic acid are improved by the modification of the base, sugar, or phosphoric acid backbone. The antisense nucleic acid may be synthesized by an ordinary method using, for example, a commercially available automatic DNA synthesizing apparatus (for example, manufactured by Applied Biosystems). The preparation of the modified nucleic acid and derivatives may refer to, for example, Stein et al. (1988), Nucl. Acids Res. 16:3209 or Sarin et al., (1988), Proc. Natl. Acad. Sci. U.S.A. 85:7448-7451.

In order to improve the action of the antisense nucleic acid in the target cell, a promoter (for example, actin promoter or ie1 promoter) that strongly acts in the target cell may be used. More specifically, when a construct containing the antisense nucleic acid under control of the promoter is introduced into the target cell, a sufficient amount of antisense nucleic acid is transcribed.

The inhibition of expression by ribozyme may be used. The mRNA corresponding to the target gene may be destroyed using a ribozyme that cleaves the mRNA at the site-specific recognition sequence, but preferably a hammerhead ribozyme is used. The method for constructing the hammerhead ribozyme may be referred to, for example, Haseloff and Gerlach, 1988, Nature, 334:585-591.

In the same manner as in the antisense method, for example, for the purpose of improving stability and target performance, the ribozyme construction may use a modified oligonucleotide. In order to produce an effective amount of ribozyme within the target cell, it is preferred that a nucleic acid construct including DNA coding the ribozyme be used under the control of a promoter which strongly acts in fungi cells (for example, an actin promoter or an ie1 promoter).

The present disclosure pertains also to transgenic plants comprising a gene coding an inhibitor against CSP of a target pathogen, wherein said inhibitor inhibits the CSP expression and/or binds to a protein product of a gene coding CSP as described herein.

Furthermore, the present disclosure relates to transgenic plants that (a) contain nucleotide sequences encoding the isolated and substantially purified nucleic acid molecules and the non-naturally occurring recombinant DNA constructs for transcribing the dsRNA molecules for controlling plant pathogen infection, and (b) display resistance and/or enhanced tolerance to the pathogen infection, are also provided. Compositions containing a dsRNA nucleotide sequences as a CSP-inhibitor according to the present disclosure for use in topical applications onto plants to achieve the elimination or reduction of the target pathogen are also described.

The term “plant” includes the plant body, plant organs (for example, leaves, petals, stem, root, rhizome, kernels and seeds), plant tissues (for example, epidermis, phloem, parenchyma, xylem, endosperm and vascular bundle), and plant cells. In addition, the term “plant cell” includes seed suspension cultures, embryos, meristematic tissue regions, callus tissues, cells derived from leaves and roots, and gametophytes (embryos and pollens) and their precursors. When plant culture cells are transformed, an organ or individual is regenerated from the transformed cells by a known tissue culture method. These operations are readily performed by those skilled in the art. An example is described below. Firstly, the transformed plant cells are cultured in a sterilized callus forming medium (containing a carbon source, saccharides, vitamins, inorganics, and phytohormones such as auxin and cytokinin), thereby forming a dedifferentiated calluse which indefinitely proliferates (callus induction). The formed callus is transferred to a new medium containing a plant growth regulator such as auxin, and further proliferated thereon (subcultivation). When the callus induction is carried out on a solid medium such as agar and subcultivation is carried out in a liquid medium, the respective cultures are efficiently achieved. Secondly, the callus proliferated by subcultivation was cultured under appropriate conditions, thereby inducing redifferentiation of the organ (inductive redifferentiation), and regenerating the plant body. The inductive redifferentiation is achieved by appropriately adjusting the type and amount of the various components of the medium, including plant growth regulators such as auxin and cytokinin, and the carbon source, and the light and temperature. The inductive redifferentiation forms adventitious embryos, adventitious roots, adventitious buds, adventitious foliage, and others, and they are grown into a complete plant body. The plant before being a complete plant body may be stored in the form of, for example, capsulated artificial seeds, dry embryos, lyophilized cells, or tissues.

In accomplishing the foregoing, the present disclosure provides methods of inhibiting expression of the CSP encoding target gene in a fungal pest, in particular in a fungus belonging to the order Eurotiales, in particular of family Trichocomaceae and genera of Aspergillus, resulting in the cessation of growth, development, reproduction, infectivity, and eventually may result in the death of the fungi.

The method comprises in one embodiment introducing partial or fully stabilized double-stranded RNA (dsRNA) nucleotide molecules derived from a CSP-encoding sequence like SEQ ID NO. 1 or SEQ ID NO. 2 into the target pathogen. Uptake of the double stranded or siRNA molecules results in the inhibition of expression of at least the CSP-encoding target gene in the cells of the pathogen. Inhibition of the target gene exerts a deleterious effect upon the pathogen.

In certain embodiments, dsRNA molecules provided by the disclosure comprise nucleotide sequences complementary to a nucleic acid sequence comprised in SEQ ID NO:1 like SEQ ID NO:2, the inhibition of which in a pathogen organism results in the reduction or removal of CSP. The nucleotide sequence selected may exhibit from about 50% to at least about 100% sequence identity to 15 to 30, in particular 27 contiguous nucleotides of SEQ ID NO:1, including the complement thereof. Such inhibition can be described as specific in that a nucleotide sequence from a portion of the CSP encoding target gene is chosen from which the inhibitory dsRNA or siRNA is transcribed. The method is effective in inhibiting the expression of the CSP target gene and can be used to inhibit many different types of pests.

In advantageous embodiments, the nucleic acid sequences identified as having a pathogen protective effect may be readily expressed as dsRNA molecules through the creation of appropriate expression constructs. For example, such sequences can be expressed as a hairpin and stem and loop structure by taking a first segment corresponding to SEQ ID NO:1 or a fragment or homolog thereof, linking this sequence to a second segment spacer region that is not homologous or complementary to the first segment, and linking this to a third segment that transcribes an RNA, wherein at least a portion of the third segment is substantially complementary to the first segment. Such a construct forms a stem and loop structure by hybridization of the first segment with the third segment and a loop structure forms comprising the second segment (WO94/01550, WO98/05770, US 2002/0048814A1, and US 2003/0018993 A1).

As mentioned above, the methods of inhibiting the growth of a target pathogen expressing the cysteine-rich secreted protein (CSP) of the present disclosure includes that the inhibitor against CSP is incorporated inside the target pathogen. In particular, the incorporated inhibitor is an RNAi inducing compound like a short interfering nucleic acids, siNA, short interfering RNA (siRNA), microRNA (miRNA), short hairpin RNAs (shRNA) and precursors thereof which are processed in the cell to the actual RNAi inducing compound. In some embodiments, the precursor is a double-stranded RNA (dsRNA), for example the dsRNA is derived from SEQ ID NO: 1, or homologs thereof, wherein said homologs have a sequence identity of at least 60%, in particular of at least 70%, in particular of at least 85%, in particular of at least 85%, in particular of at least 90%, in particular of at least 95%, in particular of at least 96, 97, 98 or 99% to SEQ ID NO: 1. In an advantageous embodiment, the dsRNA comprises SEQ ID NO: 2, or homologs thereof.

Further examples of CSP-inhibitors include also substances that specifically bind to the already expressed CSP (for example, an antibody, an antibody fragment, a peptide or a low molecular weight compound (small molecule)). The substance that specifically binds to the expressed CSP may be obtained or prepared using binding assay targeted at CSP. An antibody that specifically binds to CSP may be prepared using, for example, an immunological method, a phage display method, or a ribosome display method.

Advantageous embodiments for CSP-inhibitors that binds to a protein product of a gene coding CSP are small chemical molecule targeted the protein product of a gene coding CSP and peptides or polypeptides targeted the protein product of a gene coding CSP.

The terms “polypeptide”, “peptide”, or “protein” are used interchangeably herein to designate a linear series of amino acid residues connected one to the other by peptide bonds between the alpha-amino and carboxyl groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids.

In an advantageous embodiment, the CSP-inhibitor is an antibody or an antibody fragment selected from the group consisting of a monoclonal antibody, polyclonal antibody, Fab, scFv, single domain, or a fragment thereof, bis scFv, F(ab′)₂, F(ab)₃, minibody, diabody, triplebody, tetrabody and tandab, wherein the antibody or antibody fragment binds specifically to CSP.

An antibody is in particular specific for a particular antigen if it binds that particular antigen in preference to other antigens. In particular, the antibody may not show any significant binding to molecules other than that particular antigen, and specificity may be defined by the difference in affinity between the target antigen and other non-target antigens. An antibody may also be specific for a particular epitope which may be carried by a number of antigens, in which case the antibody will be able to bind to the various antigens carrying that epitope. For example, specific binding may exist when the dissociation constant for a dimeric complex of antibody and antigen is 1 μM, preferably 100 nM and most preferably 1 nM or lower.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these antibody light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)₂, a dimer of Fab which itself is a light chain joined to V.sub.H-C.sub.H1 by a disulfide bond. The F(ab′)₂ may be reduced under mild conditions to break the disulfide linkage in the hinge region thereby converting the F(ab′)₂ dimer into two Fab′ monomers. The Fab′ monomer is essentially a Fab with part of the hinge region (Paul 1993). While various antibody fragments are defined in terms of the digestion of an intact antibody, one of skill will appreciate that such Fab′ fragments may be synthesized de novo either chemically or by utilizing recombinant DNA technology. Thus, the term antibody, as used herein also includes antibody fragments either produced by the modification of whole antibodies or synthesized de novo using recombinant DNA technologies. Preferred antibodies include single chain antibodies (antibodies that exist as a single polypeptide chain), more preferably single chain Fv antibodies (scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker (Huston, Levinson et al. 1988). While the VH and VL are connected to each as a single polypeptide chain, the VH and VL domains associate non-covalently.

As mentioned above, the phrase “specifically binds to CSP” refers to a binding reaction, which is determinative of the presence of an antigen protein (CSP) in the presence of a heterogeneous population of proteins and other biologics. Thus, under designated immunoassay conditions, the specified antibodies or antibody fragments as CSP inhibitors according to the present disclosure bind to the expressed CSP and do not bind in a significant amount to other proteins present in the sample.

In some advantageous embodiments, the peptide and/or the polypeptide binds to CSP, in particular to a CSP comprising the amino acid sequence of SEQ ID NO:3, or homologs thereof, wherein said homologs have a sequence identity of at least 60%, in particular of at least 70%, in particular of at least 85%, in particular of at least 85%, in particular of at least 90%, in particular of at least 95%, in particular of at least 96, 97, 98 or 99% to SEQ ID NO:3 and encodes a functional CSP in the target pathogen.

Furthermore, the present disclosure relates to methods for the treatment of a disease associated with a target pathogen expressing CSP. Therefore, the above mentioned inhibitors may be used as a medicine, in particular for treatment, prevention or alleviation of a disease associated with a CSP-expressing pathogen as mentioned in the present disclosure. This can comprise the step of administering to such a living body like an animal or a human in need thereof a therapeutically effective amount of an inhibitor as described above in detail.

For example, diseases associated with an aflatoxin-producing fungus includes acute hepatic necrosis, liver damage, liver cirrhosis, liver cancer, mental impairment, abdominal pain, vomiting, convulsions, edema, pulmonary edema, hemorrhaging and disruption of food digestion, absorption a metabolism.

As mentioned above, some of the inhibitors described in the present disclosure can be used as “fungal control agent”, or “gene suppression agent”, that refers in particular to a particular RNA molecule comprising a first RNA segment and a second RNA segment, wherein the complementarity between the first and the second RNA segments results in the ability of the two segments to hybridize in vivo and in vitro to form a double stranded molecule. It may generally be preferable to include a third RNA segment linking and stabilizing the first and second sequences such that the entire structure forms into a stem and loop structure, or even more tightly hybridizing structures may form into a stem-loop knotted structure. Alternatively, a symmetrical hairpin could be formed without a third segment in which there is no designed loop, but for steric reasons a hairpin would create its own loop when the stem is long enough to stabilize itself. The first and the second RNA segments will generally be within the length of the RNA molecule and are substantially inverted repeats of each other and linked together by the third RNA segment. The first and the second segments correspond invariably and not respectively to a sense and an antisense sequence with respect to the target RNA transcribed fern the target gene in the target fungal pathogen that is suppressed by the ingestion of the dsRNA molecule. The fungal control agent can also be a substantially purified (or isolated) nucleic acid molecule and more specifically nucleic acid molecules or nucleic acid fragment molecules thereof from a genomic DNA (gDNA) or cDNA library. Alternatively, the fragments may comprise smaller oligonucleotides having from about 15 to about 250 nucleotide residues, and more preferably, about 15 to about 30 nucleotide residues.

The present disclosure provides DNA constructs, in particular DNA constructs for use in achieving stable transformation of particular host pathogen targets. Transformed host or symbiont pathogen targets may express pesticidally effective levels of preferred dsRNA or siRNA molecules from the recombinant DNA constructs, and provide the molecules to the pathogen. Pairs of isolated and purified nucleotide sequences may be provided from cDNA library and/or genomic library information. The pairs of nucleotide sequences may be for example derived from any preferred Aspergillus fungi for use as thermal amplification primers to generate DNA templates for the preparation of dsRNA and siRNA molecules of the present disclosure.

Provided according to the present disclosure are nucleotide sequences, the expression of which results in an RNA sequence which is substantially homologous to an RNA molecule of a targeted gene in an fungi that comprises an RNA sequence encoded by a nucleotide sequence within the genome of the fungi. Thus, after uptake of the stabilized RNA sequence down-regulation of the nucleotide sequence of the target gene in the cells of the fungi may be obtained resulting in a deleterious effect on the maintenance, viability, proliferation, reproduction and infection of the fungi.

Examples of isolated polynucleotide suitable as a pathogen control agent against a target pathogen expressing CSP are the following (A) to (f):
a polynucleotide derived from a nucleic acid sequence selected from the group consisting of SEQ ID NO:1 or SEQ ID NO:2;
a polynucleotide comprising a nucleic acid sequence selected from the group consisting of SEQ ID NO:1 or SEQ ID NO:2;
a polynucleotide that hybridizes to a nucleic acid sequence selected from the group consisting of SEQ ID NO:1 or SEQ ID NO:2 under stringent conditions;
a polynucleotide of at least 70, at least 80, at least 85, at least 90 percent sequence identity, to a nucleic acid sequence selected from the group consisting of SEQ ID NO:1 or SEQ ID NO:2;
a fragment of at least 16 contiguous nucleotides of a nucleic acid sequence selected from the group consisting of SEQ ID NO:1 or SEQ ID NO:2; and
a complement of the sequence of (a), (b), (c), (d) or (e).

Further provided by the disclosure is a fragment or concatemer of a nucleic acid sequence of SEQ ID NO:1. The fragment may be defined as causing the death, inhibition, stunting, or cessation of pathogen when expressed as a dsRNA and provided to the pest. The fragment may, for example, comprise at least about 15, 16, 17, 18, 19, 21, 23, 25, 26, 27, 40, 60, 80, 100, 125 or more contiguous nucleotides of the sequence set force in SEQ ID NO:1, or a complement thereof. The disclosure also provides a ribonucleic acid expressed from any of such sequences including a dsRNA. A sequence selected for use in expression of a gene suppression agent can be constructed from a single sequence derived from one or more target pests and intended for use in expression of an RNA that functions in the suppression of a single gene or gene family in the one or more target pathogen, or that the DNA sequence can be constructed as a chimera from a plurality of DNA sequences.

In further embodiments, the disclosure pertains to recombinant DNA constructs comprising a nucleic acid molecule encoding a dsRNA molecule described herein. The dsRNA may be formed by transcription of one strand of the dsRNA molecule from a nucleotide sequence which is at least from about 80% to about 100% identical to a nucleotide sequence comprising SEQ ID NO:1 or derived from SEQ ID NO:1 like SEQ ID NO:2. Such recombinant DNA constructs may be defined as producing dsRNA molecules capable of inhibiting the expression of endogenous target gene(s) in a pathogen cell upon uptake. The construct may comprise a nucleotide sequence of the plant operably linked to a promoter sequence that functions in the host cell. Such a promoter may be tissue-specific and may, for example, be specific to a tissue type which is the subject of fungal attack. In the case of maize infection, for example, it may be desired to use a promoter providing seed-preferred expression.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, and polyadenylation recognition sequences and the like.

The term “plasmid”, “vector system”, “vector” or “expression vector” means a construct capable of in vivo or in vitro expression. In the context of the present disclosure, these constructs may be used to introduce genes encoding enzymes into host cells.

The term “polynucleotide” corresponds to any genetic material of any length and any sequence, comprising single-stranded and double-stranded DNA and RNA molecules, including regulatory elements, structural genes, groups of genes, plasmids, whole genomes and fragments thereof.

The term “recombinant DNA” or “recombinant nucleotide sequence” refers to DNA that contains a genetically engineered modification through manipulation via mutagenesis, restriction enzymes, and the like.

The term “stringent conditions” relates to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion (or other salts) at pH 7.0 to 8.3 and the temperature is at least about 30° C. for short probes (e.g. 10 to 50 nucleotides) and at least about 60° C. for longer probes. Stringent conditions may also be achieved with the addition of destabilizing agents, such as formamide and the like.

Nucleic acid constructs in accordance with the disclosure may comprise at least one non-naturally occurring nucleotide sequence that can be transcribed into a single stranded RNA capable of forming a dsRNA molecule in vivo through hybridization. Such dsRNA sequences self-assemble and can be provided to achieve the desired inhibition.

A recombinant DNA construct may comprise two different non-naturally occurring sequences which, when expressed in vivo as dsRNA sequences and provided in the diet of a target pathogen, inhibit the expression of at least two different target genes in the cell of the target pathogen. In certain embodiments, at least 3, 4, 5, 6, 8 or 10 or more different dsRNAs are produced in a cell or plant comprising the cell that has a pathogen-inhibitory effect. The dsRNAs may expressed from multiple constructs introduced in different transformation events or could be introduced on a single nucleic acid molecule. The dsRNAs may be expressed using a single promoter or multiple promoters.

The present disclosure provides DNA sequences capable of being expressed as an RNA in a cell or microorganism to inhibit target gene expression. The sequences may comprise a DNA molecule coding for one or more different nucleotide sequences, wherein each of the different nucleotide sequences comprises a sense nucleotide sequence and an antisense nucleotide sequence connected by a spacer sequence coding for a dsRNA molecule of the present disclosure. The spacer sequence may constitute part of the sense nucleotide sequence or the antisense nucleotide sequence and forms within the dsRNA molecule between the sense and antisense sequences. The sense nucleotide sequence or the antisense nucleotide sequence may be substantially identical to the nucleotide sequence of the target gene or a derivative thereof or a complementary sequence thereto. The dsDNA molecule may be placed operably under the control of a promoter sequence that functions in the cell, tissue or organ of the host expressing the dsDNA to produce dsRNA molecules. In one embodiment, the DNA sequence may be derived from a nucleotide sequence of SEQ ID NO:1.

As mentioned above, the present disclosure also provides a DNA sequence for expression in a cell of a plant that, upon expression of the DNA to RNA and uptake by a target fungal pathogen achieves suppression of a target gene in a cell of the target pathogen. The dsRNA at least comprises one or multiple structural gene sequences, wherein each of the structural gene sequences comprises a sense nucleotide sequence and an antisense nucleotide sequence connected by a spacer sequence that forms a loop within the complementary and antisense sequences. The sense nucleotide sequence or the antisense nucleotide sequence is substantially identical to the nucleotide sequence of the target gene, derivative thereof, or sequence complementary thereto. The one or more structural gene sequences is placed operably under the control of one or more promoter sequences, at least one of which is operable in the cell, tissue or organ of a prokaryotic or eukaryotic organism, particularly a plant.

A gene sequence or fragment for pest control according to the present disclosure may be cloned between two tissue specific promoters, such as two seed or root specific promoters which are operable in a transgenic plant cell and therein expressed to produce rnRNA in the transgenic plant cell that form dsRNA molecules thereto. The dsRNA molecules contained in plant tissues are uptaken by a target pathogen so that the intended suppression of the CSP gene expression is achieved.

A nucleotide sequence provided by the present disclosure may comprise an inverted repeat separated by a “spacer sequence.” The spacer sequence may be a region comprising any sequence of nucleotides that facilitates secondary structure formation between each repeat, where this is required. In one embodiment of the present disclosure, the spacer sequence is part of the sense or antisense coding sequence for mRNA. The spacer sequence may alternatively comprise any combination of nucleotides or homologues thereof that are capable of being linked covalently to a nucleic acid molecule. The spacer sequence may comprise a sequence of nucleotides of at least about 10-100 nucleotides in length, or alternatively at least about 100-200 nucleotides in length, at least 200-400 about nucleotides in length, or at least about 400-500 nucleotides in length.

The nucleic acid molecules or fragment of the nucleic acid molecules or other nucleic acid molecules in the sequence listing are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook et al. (1989), and by Haymes et al. (1985).

Departures from complete complementarity are therefore permissible, as long as such departures do not completely preclude the capacity of the molecules to form a double- stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed.

Appropriate stringency conditions which promote DNA hybridization are, for example, 6.0×sodium chloride/sodium citrate (SSC) at about 45° C., followed by a wash of 2.0×SSC at 50° C., are known to those skilled in the art or can be found in Current Protocols in Molecular Biology (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. A nucleic acid for use in the present disclosure may specifically hybridize to one or more of nucleic acid molecules from Aspergillus or complements thereof under such conditions. Preferably, a nucleic acid for use in the present disclosure will exhibit at least from about 85%, or at least from about 90%, or at least from about 95%, or at least from about 98% or even about 100% sequence identity with a nucleic acid molecule of SEQ ID NO:1 or is derived from SEQ ID NO:1 like SEQ ID NO:2.

Nucleic acids of the present disclosure may also be synthesized, either completely or in part, especially where it is desirable to provide plant-preferred sequences, by methods known in the art. Thus, all or a portion of the nucleic acids of the present disclosure may be synthesized using codons preferred by a selected host. Species-preferred codons may be determined, for example, from the codons used most frequently in the proteins expressed in a particular host species. Other modifications of the nucleotide sequences may result in mutants having slightly altered activity. dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition. In one embodiment, the dsRNA molecules may be modified through an enzymatic process so that siRNA molecules may be generated. The siRNA can efficiently mediate the down-regulation effect for some target genes in some fungi. This enzymatic process may be accomplished by utilizing an RNAse III enzyme or a DICER enzyme, present in the cells of an insect, a vertebrate animal, a fungus or a plant in the eukaryotic RNAi pathway (Elbashir et al., 2002; Hamilton and Baulcombe, 1999). This process may also utilize a recombinant DICER or RNAse III introduced into the cells of a target fungi through recombinant DNA techniques that are readily known to the skilled in the art. Both the DICER enzyme and RNAse III, being naturally occurring in an fungi or being made through recombinant DNA techniques, cleave larger dsRNA strands into smaller oligonucleotides. The DICER enzymes specifically cut the dsRNA molecules into siRNA pieces each of which is about 19-25 nucleotides in length while the RNAse III enzymes normally cleave the dsRNA molecules into 12-15 base-pair siRNA. The siRNA molecules produced by the either of the enzymes have 2 to 3 nucleotide 3′ overhangs, and 5′ phosphate and 3′ hydroxyl termini. The siRNA molecules generated by RNAse III enzyme are the same as those produced by DICER enzymes in the eukaryotic RNAi pathway and are hence then targeted and degraded by an inherent cellular RNA-degrading mechanism after they are subsequently unwound, separated into single-stranded RNA and hybridize with the RNA sequences transcribed by the target gene. This process results in the effective degradation or removal of the RNA sequence encoded by the nucleotide sequence of the target gene in the fungi. The outcome is the silencing of a particularly targeted nucleotide sequence within the fungi. Detailed descriptions of enzymatic processes can be found in Harmon (2002).

The manner for incorporation of a CSP inhibitor is not particularly limited, and may be selected according to the target pathogen. When the target pathogen is a fungi that attacks a plant, for example, the agent (pesticide) containing the CSP inhibitor is in advance retained in the plant, which is to be attacked by the target pathogen, through application, spraying, or atomization. As a result of this, when the target pathogen infect the plant, the CSP inhibitor is incorporated inside of the target pathogen.

On the other hand, when the CSP inhibitor is placed at the site of occurrence or in the route of entry of the target fungal spores, the target pathogen will uptake the CSP inhibitor and pathogen infection. In addition, when the plant to be attacked is modified by the introduction of a gene coding the CSP inhibitor, the CSP inhibitor is incorporated inside the target pathogen when the pathogen infect the transgenic plant. The transgenic plant used in this method may be a plant subjected to gene modification so as to express: (A) an siRNA targeted at a gene coding the CSP of the target pest; (B) an antisense nucleic acid targeted at the transcript product of a gene coding the CSP of the target pest; or (C) a ribozyme targeted at the transcript product of a gene coding the CSP of the target pest.

Therefore, in some embodiments, the pathogen control method according to the present disclosure comprise making a plant, which is to be attacked by the target pathogen, possess an agent containing the inhibitor by application, spraying, or atomization in advance, and incorporating the inhibitor inside of the target pathogen by infection.

However, in some advantageous embodiments, the pathogen control method according to the present disclosure comprises incorporating the inhibitor into the body of the target pest by ingestion of a transgenic plant containing a gene encoding the inhibitor.

As mentioned above, the present disclosure contemplates transformation of a nucleotide sequence of the present disclosure into a plant to achieve pathogen inhibitory levels of expression of one or more dsRNA molecules. A transformation vector can be readily prepared using methods available in the art. The transformation vector comprises one or more nucleotide sequences that is/are capable of being transcribed to an RNA molecule and that is/are substantially homologous and/or complementary to one or more nucleotide sequences encoded by the genome of the fungi, such that upon uptake of the RNA there is down-regulation of expression of at least one of the respective nucleotide sequences of the genome of the fungi.

The transformation vector may be termed a dsDNA construct and may also be defined as a recombinant molecule, an fungi control agent, a genetic molecule or a chimeric genetic construct. A chimeric genetic construct of the present disclosure may comprise, for example, nucleotide sequences encoding one or more antisense transcripts, one or more sense transcripts, one or more of each of the aforementioned, wherein all or part of a transcript therefrom is homologous to all or part of an RNA molecule comprising an RNA sequence encoded by a nucleotide sequence within the genome of a fungus.

A plant transformation vector may contain sequences from more than one gene, thus allowing production of more than one dsRNA for inhibiting expression of two or more genes in cells of a target pathogen. One skilled in the art will readily appreciate that segments of DNA whose sequence corresponds to that present in different genes can be combined into a single composite DNA segment for expression in a transgenic plant. Alternatively, a plasmid of the present disclosure already containing at least one DNA segment can be modified by the sequential insertion of additional DNA segments between the enhancer and promoter and terminator sequences. In the fungi control agent of the present disclosure designed for the inhibition of multiple genes, the genes to be inhibited can be obtained from the same fungi species in order to enhance the effectiveness of the fungal control agent. In certain embodiments, the genes can be derived from different fungi in order to broaden the range of fungi against which the agent is effective. When multiple genes are targeted for suppression or a combination of expression and suppression, a polycistronic DNA element can be fabricated as illustrated and disclosed in Fillatti, Application Publication No. US 2004-0029283.

Promoters that function in different plant species are also well known in the art. Promoters useful for expression of polypeptides in plants include those that are inducible, viral, synthetic, or constitutive as described in Odell et al. (1985), and/or promoters that are temporally regulated, spatially regulated, and spatio-temporally regulated. Preferred promoters include the enhanced CaMV 35S promoters, the SUC2 promoter and the FMV 35S promoter.

The seed and endosperm located expression of target specific dsRNA or siRNA in genetically modified plants that targets CSP allows most likely the reduction of infestation of crop plants by fungi under the critical economic threshold. In this context varying length of dsRNA and siRNA are possible that cover different regions of CSP mRNA.

A recombinant DNA vector or construct of the present disclosure will typically comprise a selectable marker that confers a selectable phenotype on plant cells. Selectable markers may also be used to select for plants or plant cells that contain the exogenous nucleic acids encoding polypeptides or proteins of the present disclosure. The marker may encode biocide resistance, antibiotic resistance (e.g. kanamycin, G418 bleomycin, hygromycin, etc.), or herbicide resistance (e.g. glyphosate, etc.). Examples of selectable markers include, but are not limited to, a neo gene which codes for kanamycin resistance and can be selected for using kanamycin, G418, etc., a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulfonylurea resistance; and a methotrexate resistant DHFR gene. Examples of such selectable markers are illustrated in U.S. Pat. Nos. 5,550,318; 5,633,435; 5,780,708 and 6,118,047.

A recombinant vector or construct of the present disclosure may also include a screenable marker. Screenable markers may be used to monitor expression. Exemplary screenable markers include a [beta]-glucuronidase or uidA gene (GUS) which encodes an enzyme for which various chromogenic substrates are known (Jefferson, 1987; Jefferson et al., 1987); an R-locus gene, which encodes a product that regulates the production of anthocyanin pigments (red color) in plant tissues (Dellaporta et al., 1988); a [beta]-lactamase gene (Sutcliffe et al., 1978), a gene which encodes an enzyme for which various chromogenic substrates are known (e.g. PADAC, a chromogenic cephalosporin); a luciferase gene (Ow et al., 1986) a xylE gene (Zukowsky et al., 1983) which encodes a catechol dioxygenase that can convert chromogenic catechols; an [alpha]-amylase gene (Bcatu et al., 1990); a tyrosinase gene (Katz et al., 1983) which encodes an enzyme capable of oxidizing tyrosine to DOPA and dopaquinone which in turn condenses to melanin; an α-galactosidase, which catalyzes a chromogenic α-galactose substrate.

In some advantageous embodiments, the isolated polynucleotides according to the present disclosure
(i) is defined as operably linked to a heterologous promoter; or
(ii) is defined as comprised on a plant transformation vector.

In some advantageous embodiments, the isolated polynucleotides according to the present disclosure are operably linked to a heterologous promoter and/or are defined as comprised on a plant transformation vector.

Preferred plant transformation vectors include those derived from a Ti plasmid of Agrobacterium tumefaciens (e.g. U.S. Pat. Nos. 4,536,475, 4,693,977, 4,886,937, 5,501,967 and EP 0 122 791). Agrobacterium rhizogenes plasmids (or “Ri”) are also useful and known in the art. Other preferred plant transformation vectors include those disclosed, e.g. by Herrera-Estrella (1983); Bevan (1983), Klee (1985) and EP 0 120 516. In an advantageous embodiment, the vector is a binary vector.

In general it is preferred to introduce a functional recombinant DNA at a non-specific location in a plant genome. In special cases it may be useful to insert a recombinant DNA construct by site-specific integration. Several site-specific recombination systems exist which are known to function implants include cre-lox as disclosed in U.S. Pat. No. 4,959,317 and FLP-FRT as disclosed in U.S. Pat. No. 5,527,695.

Suitable methods for transformation of host cells for use with the current plant are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), by Agrobacterium-mediated transformation (U.S. Pat. No. 5,591,616 and U.S. Pat. No. 5,563,055) and by acceleration of DNA coated particles (U.S. Pat. No. 5,550,318; U.S. Pat. No. 5,538,877; and U.S. Pat. No. 5,538,880), etc. Through the application of techniques such as these, the cells of virtually any species may be stably transformed. In the case of multicellular species, the transgenic cells may be regenerated into transgenic organisms. Methods for the creation of transgenic plants and expression of heterologous nucleic acids in plants in particular are known and may be used with the nucleic acids provided herein to prepare transgenic plants that exhibit reduced susceptibility to feeding by a target pest organism such as corn rootworms. Plant transformation vectors can be prepared, for example, by inserting the dsRNA producing nucleic acids disclosed herein into plant transformation vectors and introducing these into plants. One known vector system has been derived by modifying the natural gene transfer system of Agrobacterium tumefaciens. The natural system comprises large Ti (tumor-inducing)-plasmids containing a large segment, known as T-DNA, which is transferred to transformed plants. Another segment of the Ti plasmid, the vir region, is responsible for T-DNA transfer. The T-DNA region is bordered by terminal repeats, hi the modified binary vectors the tumor-inducing genes have been deleted and the functions of the vir region are utilized to transfer foreign DNA bordered by the T-DNA border sequences. The T-region may also contain a selectable marker for efficient recovery of transgenic plants and cells, and a multiple cloning site for inserting sequences for transfer such as a dsRNA encoding nucleic acid.

A transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A homozygous transgenic plant can be obtained by sexually mating (selfmg) an independent segregant transgenic plant to produce F1 seed. One fourth of the F1 seed produced will be homozygous with respect to the transgene. Germinating F1 seed results in plants that can be tested for heterozygosity or homozygosity, typically using a SNP assay or a thermal amplification assay that allows for the distinction between heterozygotes and homozygotes (i.e. a zygosity assay).

The methods and compositions of the present disclosure may be applied to any monocot and dicot plant, depending on the aflatoxin producing fungi control desired. Specifically, the plants are intended to include, without limitation, alfalfa, aneth, apple, apricot, artichoke, arugula, asparagus, avocado, banana, barley, beans, beet, blackberry, blueberry, broccoli, brussel sprouts, cabbage, canola, cantaloupe, carrot, cassava, cauliflower, celery, cherry, cilantro, chilly, citrus, Clementine, coffee, corn, cotton, cucumber, Douglas fir, eggplant, endive, escarole, eucalyptus, fennel, figs, gourd, grape, grapefruit, honey dew, jicama, kiwifruit, lettuce, leeks, lemon, lime, Loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini plants. Thus, a plant transformed with a recombinant DNA sequence of SEQ ID NO:1, or concatemer, fragment, or complement thereof, that is transcribed to produce at least one dsRNA molecule that functions when ingested by a fungi to inhibit the expression of a target gene in the pathogen is also provided by the plant. In particular embodiments, the recombinant DNA sequence is SEQ ID NO:2, or fragments, complements, or concatemers thereof.

However, the polynucleotide according to the present disclosure may be transformed, transduced or transfected via a recombinant DNA vector also in a prokaryotic cell or eukaryotic cell, for example for production of an agent (pesticide) containing the CSP inhibitor.

A recombinant DNA vector may, for example, be a linear or a closed circular plasmid. The vector system may be a single vector or plasmid or two or more vectors or plasmids that together contain the total DNA to be introduced into the genome of the bacterial host. In addition, a bacterial vector may be an expression vector. The nucleic acid molecules according to the present disclosure can, for example, be suitably inserted into a vector under the control of a suitable promoter that functions in one or more microbial hosts to drive expression of a linked coding sequence or other DNA sequence. Many vectors are available for this purpose, and selection of the appropriate vector will depend mainly on the size of the nucleic acid to be inserted into the vector and the particular host cell to be transformed with the vector. Each vector contains various components depending on its function (amplification of DNA or expression of DNA) and the particular host cell with which it is compatible. The vector components for bacterial transformation generally include, but are not limited to, one or more of the following: a signal sequence, an origin of replication, one or more selectable marker genes, and an inducible promoter allowing the expression of exogenous DNA.

Some embodiments pertain to isolated and purified nucleotide sequences as CSP inhibitors that may be used as the pathogen, in particular fungal control agents.

Therefore, the present disclosure provides a method for obtaining a nucleic acid comprising a nucleotide sequence for producing a dsRNA or siRNA. In one embodiment, such a method for obtaining a nucleic acid fragment comprises a nucleotide sequence for producing a substantial portion of a dsRNA or siRNA comprises: (a) synthesizing first and a second oligonucleotide primers corresponding to a portion of one of the nucleotide sequences from a targeted pathogen; and (b) amplifying a cDNA or gDNA template in a cloning vector using the first and second oligonucleotide primers of step (a) wherein the amplified nucleic acid molecule transcribes a substantial portion of a dsRNA or siRNA of the present invention. The preferred target genes of the present disclosure are genes encoding CSP. In one embodiment, a gene is selected that is expressed inside the fungi.

For the purpose of the present invention, the dsRNA or siRNA molecules may be obtained from a CSP encoding DNA or RNA by polymerase chain (PCR) amplification of a target CSP gene sequences.

Nucleic acid molecules and fragments thereof from Aspergillus species, or other aflatoxin-producing pathogens like fungi may be employed to obtain other nucleic acid molecules from other species for use in the present disclosure to produce desired dsRNA and siRNA molecules. Such nucleic acid molecules include the nucleic acid molecules that encode the complete coding sequence of a protein and promoters and flanking sequences of such molecules. In addition, such nucleic acid molecules include nucleic acid molecules that encode for gene family members. Such molecules can be readily obtained by using the above-described nucleic acid molecules or fragments thereof to screen, for instance, cDNA or gDNA libraries. Methods for forming such libraries are well known in the art.

In order to obtain a DNA segment from the corresponding CSP gene in an aflatoxin-producing fungal species, PCR primers may be designed based on the sequence as found in the fungus from which the CSP gene has been cloned. The primers may be designed to amplify a DNA segment of sufficient length for use in the present disclosure. DNA (either genomic DNA or cDNA) may be prepared from the fungal species, and the CSP-specific PCR primers may be used to amplify the DNA segment. Amplification conditions may be selected so that amplification will occur even if the primers do not exactly match the target sequence. Alternately, the gene (or a portion thereof) may be cloned from a gDNA or cDNA library prepared from the fungal pathogen species, using the CSP gene or another known fungal gene as a probe. Techniques for performing PCR and cloning from libraries are known. Further details of the process by which DNA segments from target fungal pathogen species may be isolated based on the sequence of the CSP genes. One of ordinary skill in the art will recognize that a variety of techniques may be used to isolate gene segments from fungal pest species that correspond to genes previously isolated from other species.

The described agro-biotechnological approach of HIGS of CSP in crops (e.g. corn, cotton, wheat, peanut), where aflatoxin-producing fungi of genera Aspergillus are relevant pathogens, can be used to control these in the field as well as in the greenhouse. The development of resistances by fungal pathogens can be excluded on the current state of knowledge. Off-target effects on other fungi can actually be excluded because no hits were detected by BLAST search in mRNA sequences of available organisms.

Therefore, as mentioned above some embodiments pertain to plants transformed, transduced or transfected with a polynucleotide according to the present disclosure. In particular, said polynucleotide is expressed in a cell of the plant as a double stranded ribonucleotide sequence and uptake of a target pathogen inhibitory amount of said double stranded ribonucleotide sequence and/or of an RNAi inducing compound derived from said double stranded ribonucleotide sequence inhibits the target pathogen from further infections, preferably
i) the target pathogen is an aflatoxin-producing fungus belonging to the phylum Ascomycota, in particular the belonging to the order Eurotiales, in particular belonging to the family Trichocomaceae and in particular to a fungus belonging to the genera of Aspergillus, in particular Aspergillus flavus and/or Aspergillus parasiticum and/or Aspergillus fumigatus,
ii) uptake of the target pathogen inhibitory amount of the double stranded ribonucleotide sequence or fragments thereof stunts the growth of the aflatoxin-producing pathogenic fungi.

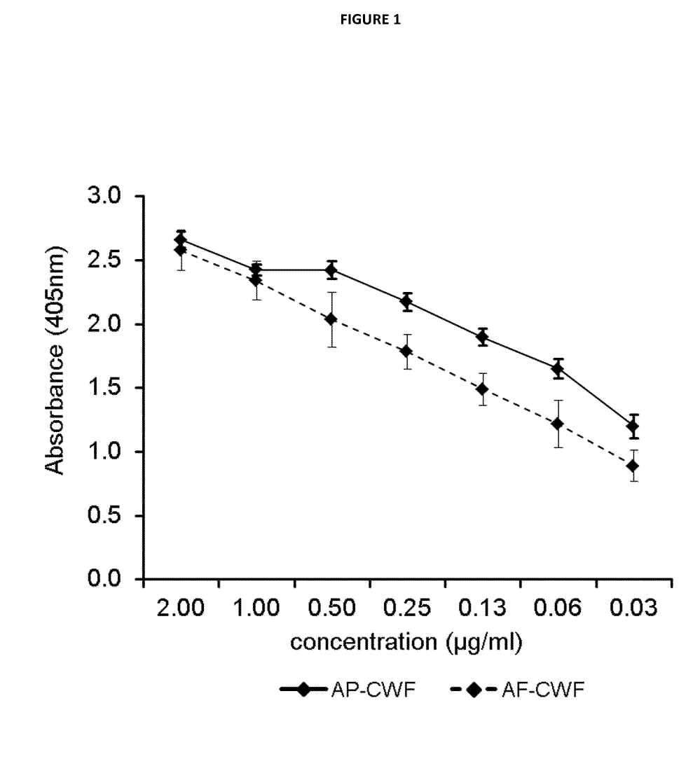

FIG. 1 shows the specific reactivity of CSP-specific mAbAP10 against A. flavus and A. parasiticus cell wall fragments as determined by ELISA. Each 200-μg sample of freeze-dried cell wall fragments from A. flavus (AF-CWF) and A. parasiticus (AP-CWF) was coated in triplicate onto the wells of high-binding microtiter plates. Aspergillus-specific mAbAP10 was added to the wells as serial dilutions from 0.0315 to 2 μg/ml, and binding was detected using a horseradish peroxidase-labeled goat anti-mouse secondary antibody. The absorbance (OD₄₀₅ₙₘ) was measured after incubation for 30 min with the substrate ABTS.

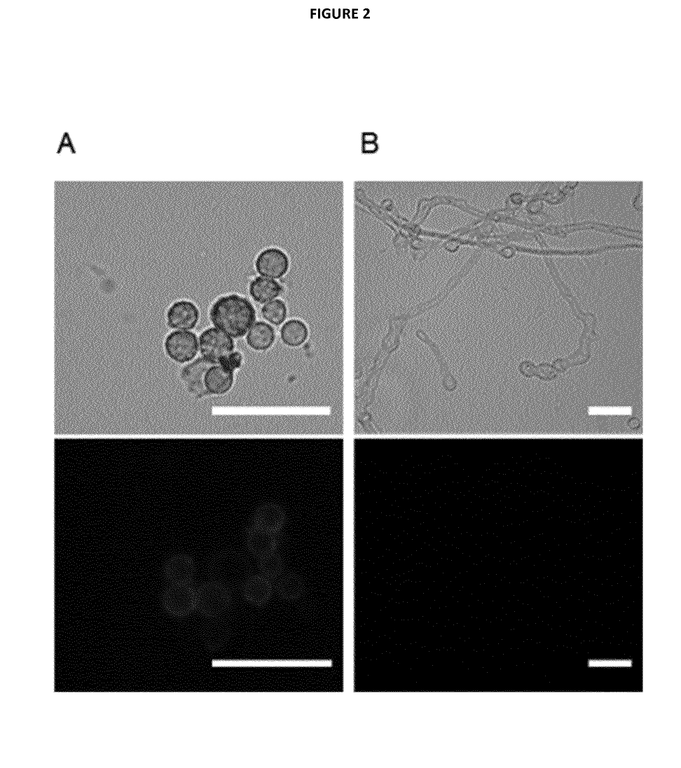

FIG. 2 shows the indirect binding of CSP-specific mAbAP10 to freshly-harvested A. flavus conidia (A) and mycelia germinated overnight (B) visualized by immunofluorescence microscopy. The specific binding of mAbAP10 to the spore surface but not to the germinated mycelia indicates the localization of CSP on the fungal surface only at the early stages of development.

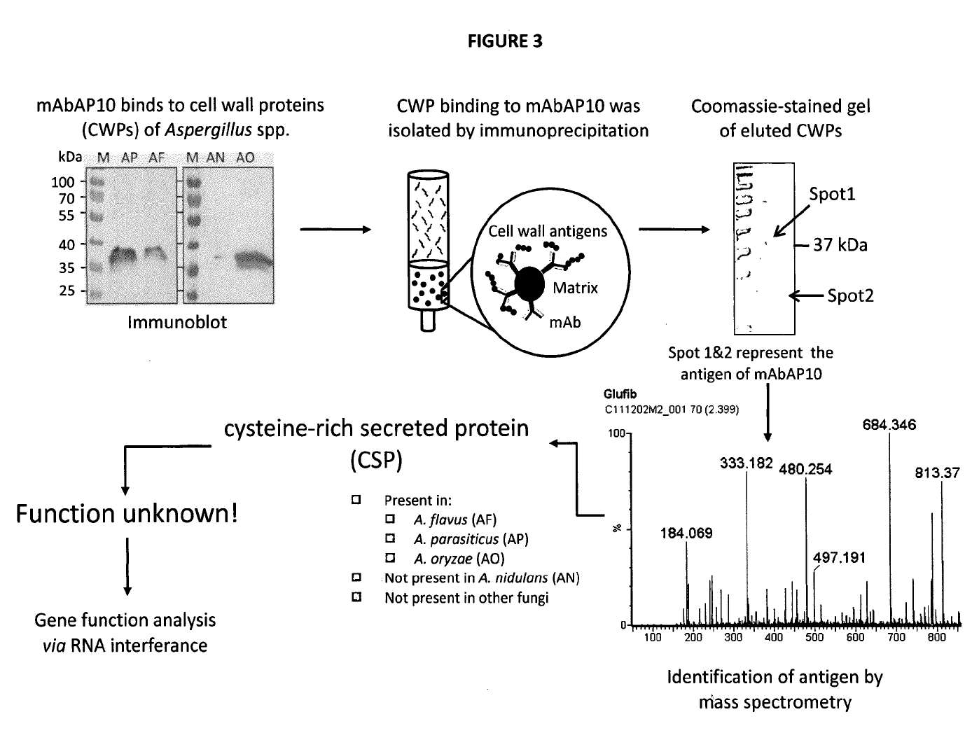

FIG. 3 presents an overview of the method used to identify the cysteine-rich secreted protein (CSP).

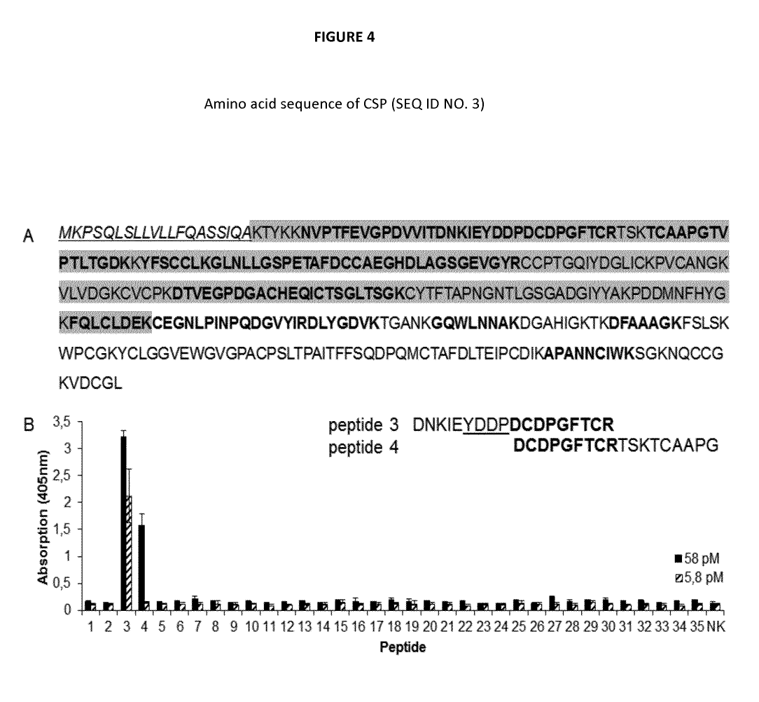

FIG. 4 shows the amino acid sequence (A) of CSP (SEQ ID NO. 3) and (B) the epitope recognized by mAbAP10.

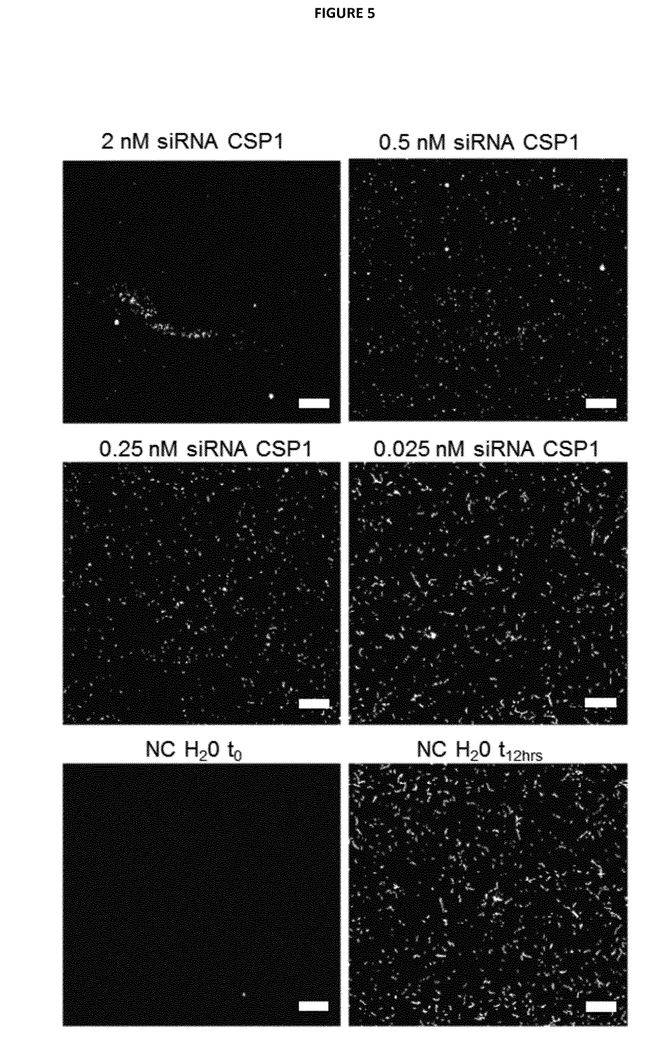

FIG. 5 shows the dose-dependent effect of CSP silencing using dsRNA derived from SEQ ID NO.1 (namely SEQ ID NO. 2) on the growth of A. flavus, compared to a water-only control. Serial dilutions (from 0.025 to 4 nM) of CSP-specific siRNA (or the water-only control) were incubated with 200 A. flavus conidia for 12 h at 28° C. in the dark. Mycelia stained with calcofluor white were visualized using the Opera® High Content Screening confocal microscope. Scale bars=100 μm.

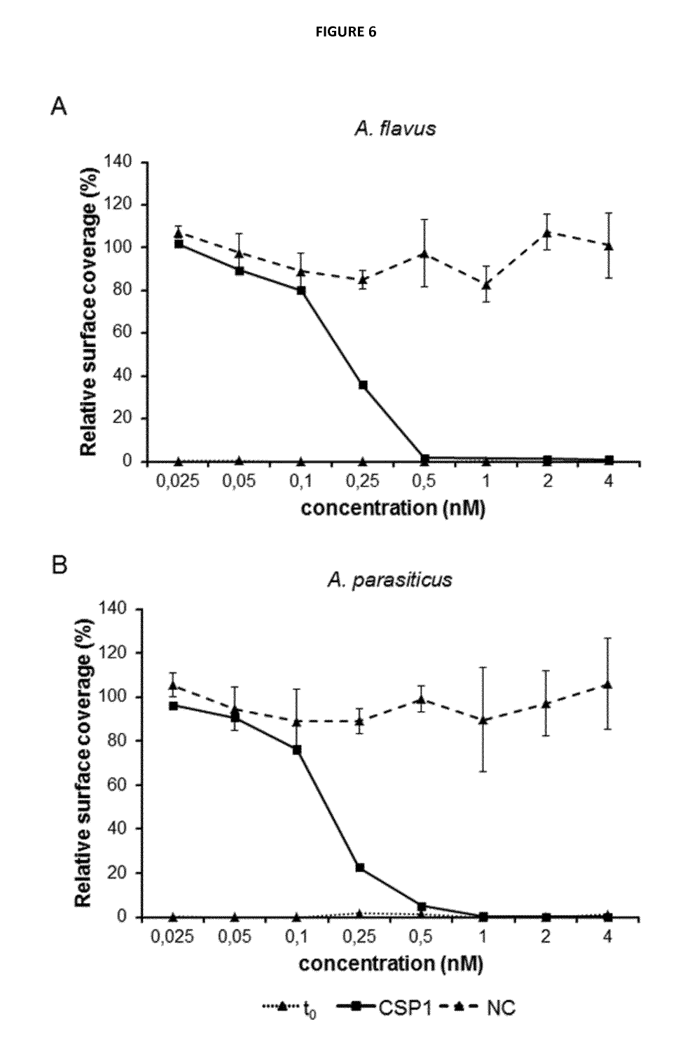

FIG. 6 shows the quantitative growth inhibition achieved by silencing with CSP-specific siRNA (SEQ ID NO. 2) in A. flavus (A) and A. parasiticus (B). The reduction of fungal growth following incubation with CSP-specific siRNA was statistically significant.

Table 1 summarizes the cross reactivity of Aspergillus-specific mAbAP10 against the cell wall proteins of several fungal pathogens, as determined by ELISA. High-binding microtiter plates were coated with 250 μg cell wall fragments per well to determine the reactivity of mAbAP10 (200 ng/ml), and a horseradish peroxidase-labeled goat-anti mouse specific secondary antibody was used for detection. The absorbance was measured after 20 min incubation with the substrate ABTS. The reactivity is classified as follows: +++>1.5, ++1.0-1.49, +0.5-0.99, 0 0.2-0.49, −<0.1. PBS was used as the negative control.

### Methods and Examples

The following examples provide the materials and methods of the present disclosure, including the determination of the effect of CSP silencing on fungal growth. These examples are for illustrative purpose only and are not to be construed as limiting this disclosure in any manner. All publications, patents, and patent applications cited herein are hereby incorporated by reference in their entirety for all purposes.

### Example 1

### Fungal Isolates and Antigen Preparation

The Aspergillus strains used in this example were Aspergillus flavus Link:Fr DSMZ 818, Aspergillus parasiticus Speare DSM1300, Aspergillus nidulans DSMZ820 and Aspergillus oryzae DSMZ1862. The strains were sub-cultured on potato dextrose agar (PDA; Carl Roth, Karlsruhe, Germany) or liquid potato dextrose broth medium (PDB; Carl Roth) for 7 days at 28° C. in the dark. For antigen preparation, Aspergillus conidia were harvested from an overgrown PDA plate as previously described (Schubert et al., 2010), cultivated in PDB medium for 7-9 days at 28° C., and the fungal material was harvested by pouring the medium through a layer of sterile miracloth (Merck Millipore, Darmstadt, Germany). The recovered material was ground in liquid nitrogen and cell wall fragments (CWFs) were prepared as previously described (Peschen et al., 2004). To obtain soluble proteins, cell wall-bound proteins were extracted from CWFs using a reducing buffer containing SDS/DTT as previously described (Pitarch et al., 2008). Finally the extracted protein was precipitated in acetone (Botelho et al., 2010) and resuspended in phosphate buffered saline (PBS). The protein was passed through a 0.45-μm filter and the amount of SDS in the precipitated samples was measured as described by Arand et al. (1992) before storage at −20° C.

### Example 2

### Generation and Characterization of an Aspergillus-Specific Antibody

The Aspergillus-specific monoclonal antibody AP10 (mAbAP10) was selected using hybridoma technology as previously described (Coligan et al., 2000; Westerwoudt, 1985). Hybridoma cultures producing mAbAP10 were settled in FCS-free Panserin® H5000 medium (PAN-Biotech, Aidenbach, Germany) and antibody production was carried out in a CELLine CL100 bioreactor (Sartorius, Aachen). The antibody mAbAP10 was purified from the enriched hybridoma supernatant using 4-mercapto-ethyl-pyridine (MEPTM-Hypercell) resin (Pall, New York, USA). The specific binding of the affinity-purified antibody to A. flavus and A. parasiticus was confirmed by ELISA (FIG. 1) and immunofluorescence microscopy (FIG. 2). A high-binding microtiter plate (Greiner Bio-One) was coated with 200 μg dry weight of AF-CWF or AP-CWF overnight at 37° C. After blocking with 3% (w/v) skimmed milk in PBS containing 0.05% (v/v) Tween-20, serial dilutions of mAbAP10 ranging from 0.031 to 2 μg/ml were loaded onto the ELISA plate. Antibody binding was detected using a goat anti-mouse Fcy antibody labeled with horseradish peroxidase (Jackson ImmunoResearch, Suffolk, UK) followed by staining with ABTS substrate. Monoclonal antibody AP10 showed highly-specific binding to A. flavus, A. parasiticus (FIG. 1) and A. oryzae, but no binding to A. nidulans, A. niger or other fungal pathogens representing the Ascomycota, Basidiomycota and Oomycota (Table 1).

The binding of mAbAP10 to the surface of germinated spores was confirmed by immunofluorescence microscopy (FIG. 2). Round glass coverslips were treated with 0.01% (v/v) poly-L-lysine (Sigma-Aldrich) and deposited in pre-blocked 12-well tissue culture plates for antigen coating (Greiner Bio-One GmbH). A 1-ml aliquot of freshly prepared A. parasiticus mycelia or germinated spores was transferred onto the coverslips and the culture plate was centrifuged (2000×g, 15 min, room temperature) to ensure firm coating. In the next step, 2 μg/ml of mAbAP10 purified from the culture supernatant was added to the wells, and binding was detected using Dylight 568-labeled goat anti-mouse Fc antibody (Invitrogen, Leek, Netherlands). The results were recorded with a Leica DMR fluorescence microscope fitted with an oil immersion objective (HCX PL APO 100×/1.40 oil PH 3 CS) and connected to a Leica DFC320 camera (Leica Microsystems Heidelberg GmbH, Mannheim, Germany). This analysis demonstrated that mAbAP10 bound strongly to the conidial cell walls but not to the surface of the germinated hyphae, indicating that mAbAP10 binds to the fungal surface specifically during the early stages of development.

### Example 3

### Identification of the mAbAP10 Antigen

The antigen recognized by mAbAP10 was characterized by separating Aspergillus cell wall proteins by SDS-PAGE (12% (w/v) polyacrylamide) and blotting them onto a nitrocellulose membrane, followed by immunodetection using affinity-purified mAbAP10 and detection using a goat anti-mouse antibody conjugated to alkaline phosphatase (Jackson ImmunoResearch). Antigen-antibody complexes were detected using substrate NBT/BCIP. This demonstrated that mAbAP10 binds specifically to a 37-kDa protein in the CWFs of A. flavus and A. parasiticus.

Immunoaffinity chromatography was carried out to enrich and/or purify the abovementioned antigen from other fungal proteins. Therefore, 80 ml of the hybridoma supernatant producing >1 mg/ml mAbAP10 was applied to an MEP HyperCell™ chromatography column, and 70 mg of the recovered antibody was coupled to NHS-activated Sepharose (GE Healthcare, Solingen, Germany) with a coupling efficiency of 99%. The soluble fungal cell wall proteins were extracted from 10 g fresh A. parasiticus CWFs, boiled in reducing extraction buffer and precipitated in acetone. The soluble cell wall proteins were applied to the column containing mAbAP10-coupled Sepharose. The bound proteins were eluted in reducing buffer containing SDS. The eluted protein fraction was separated by SDS-PAGE, and four major protein bands were observed when the gel was stained with Coomassie Brilliant Blue. In the corresponding immunoblot, the abovementioned 37-kDa full-size protein was detected by mAbAP10 as well as a smaller 22-kDa protein. Mass spectrometry showed that both proteins recognized by mAbAP10 contained peptides that matched the cysteine-rich secreted protein (CSP) from A. flavus (gi|238486514). The 338-amino-acid CSP has a low pl of 5.46 and was first described as a protein with unknown function (Payne et al., 2006). It has 99% sequence identity to a hypothetical protein sequence from A. oryzae (AOR_1_986114, gi317149420) explaining the cross-reactivity in the immunoblot (data not shown). The strategy used to identify the mAbAP10 antigen is summarized in FIG. 3.

The overlay of peptides identified by mass spectrometry and common to both spots suggests that the CSP epitope recognized by mAbAP10 is found at the N-terminus. To confirm the identity of the antigen, the CSP DNA sequence was chemically synthesized and inserted at the NcoI/NotI cloning site upstream of the His-tag sequence in vector pET-22b(+) (Merck Millipore, Darmstadt, Germany). The expression of recombinant CSP in E. coli BL21 cells, and the subsequent detection of His-tagged CSP by mAbAP1, confirmed the mass spectrometry results (data not shown).

The CSP epitope recognized by mAbAP10 was also identified by Pepscan-ELISA. As anticipated, the epitope is linear and is located close to the N-terminus. Because peptides 3 and 4 overlap and cover amino acids 25-37 and 29-46, respectively, the epitope sequence must contain the nine amino acids common to both peptides (DCDPGFTCR). However, this probably needs to be extended by 3-4 residues in the N-terminal direction (YDDP) because peptide 3 binds to the antibody with twice the efficiency of peptide 4.

### Example 4

### dsRNA Production and Uptake

To investigate the function of CSP (gi|238486514) in A. flavus and A. parasiticus, a 27mer Dicer-substrate RNA duplex (CSP1 siRNA) was chemically synthesized to silence the corresponding gene. The siRNA had a single 2-base overhang on the antisense strand and corresponds to positions 365-392 bp within the CSP gene. The siRNA was specific for the CSP gene according to the manufacturer's algorithm (Eurofins MWG Operon, Ebersberg bei Munchen, Germany). The sequence of the CSP1 siRNA was designed so that it did not overlap with any other gene by more than 20 bp, to avoid off-target effects. A siRNA representing the A. flavus antigenic cell wall mannoprotein was used as a control.

Pre-blocked black-walled 96-well microtiter plates (Greiner Bio-one) were coated with 80 μl Aspergillus spores (4×10⁴/ml) diluted 1:5 in RPMI medium, and 20 μl of the CSP1 siRNA was applied in serial dilutions ranging from 0.025 to 4 nM. The spores were incubated for 14 h at 28° C. and fungal growth was monitored using the Perkin Elmer Opera® High Content Screening System (emission 440 nm/absorption 355 nm) by staining the cell walls with 20 μl calcofluor white (diluted 1:20 in water) for 10 min at room temperature. The raw data were analyzed using Image) (NIH, Bethesda, USA). To exclude non-related image aggregates, the fungal area was calculated using a particle size (p×2) of 20-∞ and a circularity of 0.0-0.5.

Representative images demonstrated the dose-dependent inhibition of A. flavus growth using 0.025-4 nM CSP1 siRNA. A. flavus spore germination was observed in the presence of 0.5 nM CSP1 but negligible conidial growth was observed (FIG. 5). Normal germination occurred in the presence of 0.025 CSP1 siRNA after 12 h, relative to the water-only control (FIG. 5). Similar results were observed for A. parasiticus (data not shown), confirming the inhibitory effect of the CSP-specific siRNA on the growth of both species.

The inhibition of both species by CSP1 siRNA indicates that CSP plays an important role in the life cycle of A. flavus and A. parasiticus. The half maximal inhibitory concentration (IC5₀) of CSP1 was calculated based on the inhibition curves. For A. flavus, the IC₅₀ was 0.2 nM and complete inhibition of fungal growth occurred at 0.5 nM CSP1 (FIG. 6A). For A. parasiticus, the IC₅₀ was 0.175 nM and complete inhibition of fungal growth occurred at 1 nm CSP1 (FIG. 6B).

### References

Abdel-Hadi A, Carter D, Magan N (2010) Temporal monitoring of the nor-1 (afiD) gene of Aspergillus flavus in relation to aflatoxin B(1) production during storage of peanuts under different water activity levels. J Appl Microbiol 109:1914-1922. doi:10.1111/j.1365-2672.2010.04820.x

Abdel-Hadi A M, Caley D P, Carter D R, Magan N (2011) Control of aflatoxin production of Aspergillus flavus and Aspergillus parasiticus using RNA silencing technology by targeting aflD (nor-1) gene. Toxins (Basel) 3:647-659. doi:10.3390/toxins3060647

Alakonya A E, Monda E O (2013) A new approach in aflatoxin management in Africa: Targeting aflatoxin/sterigmatocystin biosynthesis in Aspergillus species by RNA silencing technique. Aflatoxins—Recent Advances and Future Prospects. doi:38211

Arand M, Friedberg T, Oesch F (1992) Colorimetric quantitation of trace amounts of sodium lauryl sulfate in the presence of nucleic acids and proteins. Anal Biochem 207:73-75.

Botelho D, Wall M J, Vieira D B, Fitzsimmons S, Liu F, Doucette A (2010) Top-down and bottom-up proteomics of SDS-containing solutions following mass-based separation. J Proteome Res 9:2863-2870. doi:10.1021/pr900949p

Brown R L, Menkir A, Chen Z Y, Bhatnagar D, Yu J, Yao H, Cleveland T E (2013) Breeding aflatoxin-resistant maize lines using recent advances in technologies—a review. Food Addit Contam 30:1382-1391. doi:10.1080/19440049.2013.812808

Coligan, J. E., A. M. Kruisbeek, D. H. Margulies, E. M. Shevach, and W. Strobel. 2000 Current Protocols in Immunology. Wiley, NY.

Kaeppler H F, Gu W, Somers D A, Rines H W, Cockburn A F. (1990) Silicon carbide fiber-mediated DNA delivery into plant cells. Plant Cell Rep. 9:415-418.

McDonald T, Brown D, Keller N P, Hammond T M (2005) RNA silencing of mycotoxin production in Aspergillus and Fusarium species. Molecular Plant-Microbe Interactactions 18:539-545. doi:10.1094/MPMI-18-0539

Omirulleh S, Abraham M, Golovkin M, Stefanov I, Karabaev M K, Mustárdy L, Morocz S, Dudits D. (1993) Activity of a chimeric promoter with the doubled CaMV 35S enhancer element in protoplast-derived cells and transgenic plants in maize. Plant Mol. Biol. 21:415-28.

Payne, G. A., Nierman, W. C., Wortman, J. R., Pritchard, B. L., Brown, D., Dean, R. A., Bhatnagar, D., Cleveland, T. E., Machida, M., Yu, J. (2006) Whole genome comparison of Aspergillus flavus and A. oryzae. Medical Mycology 44:9-11. doi:10.1080/13693780600835716

Peschen D, Li H P, Fischer R, Kreuzaler F, Liao Y C (2004) Fusion proteins comprising a Fusarium-specific antibody linked to antifungal peptides protect plants against a fungal pathogen. Nature Biotechnology 22:732-738. doi:10.1038/nbt970

Pitarch A, Nombela C, Gil C (2008) Cell wall fractionation for yeast and fungal proteomics. Methods in Molecular Biology 425:217-239. doi:10.1007/978-1-60327-210-0_19

Potrykus I, Paszkowski J, Saul M W, Petruska J, Shillito R D. (1985) Molecular and general genetics of a hybrid foreign gene introduced into tobacco by direct gene transfer. Mol Gen Genet. 199:169-77.

Schubert M, Agdour S, Fischer R, Olbrich Y, Schinkel H, Schillberg S (2010) A monoclonal antibody that specifically binds chitosan in vitro and in situ on fungal cell walls. J Microbiol Biotechnol 20:1179-1184.

Westerwoudt R J (1985) Improved fusion methods. IV. Technical aspects. J Immunol Methods 77.

1. A method of inhibiting the growth of a target pathogen expressing the cysteine-rich secreted protein (CSP), whereby the method comprises contacting said target pathogen with an inhibitor against said CSP, wherein said inhibitor inhibits the CSP expression and/or binds to a protein product of a gene coding CSP.

2. The method according to claim 1, wherein the target pathogen is an aflatoxin-producing fungus and/or a fungus belonging to the phylum Ascomycota, optionally a fungus belonging to the order Eurotiales, optionally a fungus belonging to the family Trichocomaceae, optionally a fungus belonging to the genera of Aspergillus, which is optionally selected from the group consisting of Aspergillus flavus, Aspergillus parasiticus and Aspergillus fumigatus.

3. The method according to claim 1, wherein the CSP is coded by an mRNA comprising SEQ ID NO: 1, or a homolog thereof, homolog having a sequence identity of at least 60%, optionally at least 70%, optionally at least 80%, optionally at least 85%, optionally at least 90%, optionally at least 95%, optionally at least 96, 97, 98 or 99% to SEQ ID NO: 1 and encodes a functional CSP in the target pathogen.

4. The method according to claim 1, wherein the inhibitor is a compound selected from the group consisting of the following (a) to (f):
(a) an RNAi inducing compound targeted a nucleic acid coding CSP or parts thereof;
(b) a nucleic acid construct intracellularly producing an RNAi inducing compound targeted a nucleic acid coding CSP or parts thereof;
(c) an antisense nucleic acid targeted at the transcript product of a gene coding CSP or parts thereof;
(d) a ribozyme targeted at the transcript product of a gene coding CSP or parts thereof;
(e) a small chemical molecule targeted the protein product of a gene coding CSP;
(f) a peptide or polypeptide targeted the protein product of a gene coding CSP.

5. The method according to claim 1, wherein the inhibitor is incorporated inside the target pathogen.

6. The method according to claim 4, wherein the RNAi inducing compound is a compound selected from the group consisting of a short interfering nucleic, acid (siNA), a short interfering RNA (siRNA), a microRNA (miRNA), a short hairpin RNA (shRNA) and a precursor thereof which is processed in the cell to the actual RNAi inducing compound.

7. The method according to claim 6, wherein the precursor is double-stranded RNA (dsRNA).

8. The method according to claim 7, wherein the dsRNA is derived from SEQ ID NO:1, or a homolog thereof, optionally said homolog as a sequence identity of at least 60%, optionally of at least 70%, optionally at least 80%, optionally at least 85%, optionally at least 90%, optionally at least 95%, optionally at least 96, 97, 98 or 99% to SEQ ID NO:1.

9. The method according to claim 4, wherein the dsRNA comprises SEQ ID NO:2, or a homolog thereof, preferably optionally said homolog having a sequence identity of at least 60%, optionally of at least 70%, optionally of at least 80%, optionally of at least 85%, at least 90%, optionally of at least 95%, optionally of at least 96, 97, 98 or 99% to SEQ ID NO:2.

10. The method according to claim 4, wherein the polypeptide is an antibody, or like an antibody fragment selected from the group consisting of a Fab, a scFv, a single domain, or a fragment thereof, a bis scFv, F(ab′)2, a F(ab′)3, a minibody, a diabody, a triabody, a tetrabody and a tandab.

11. The method according to claim 4 wherein the peptide and/or the polypeptide binds to CSP, optionally to a protein comprising the amino acid sequence of SEQ ID NO:3, or a homolog thereof, optionally said homolog having a sequence identity of at least 60%, optionally of at least 70%, optionally of at least 80%, optionally of at least 85%, optionally of at least 90%, optionally of at least 95%, optionally of at least 96, 97, 98 or 99% to SEQ ID NO:3.

12. The method according to claim 11, wherein the method is used for the control and/or treatment of aflatoxin-producing fungi in agriculture.

13. The method according to claim 1, wherein the method is used for the treatment of a disease associated with a pathogen expressing CSP.

14. An isolated polynucleotide selected from the group consisting of:
a) a polynucleotide derived from a nucleic acid sequence selected from SEQ ID NO:1 or SEQ ID NO:2;
b) a polynucleotide comprising a nucleic acid sequence of SEQ ID NO:1 or SEQ ID NO:2;
c) a polynucleotide that hybridizes to a nucleic acid sequence of SEQ ID NO:1 or SEQ ID NO:2 under stringent conditions;
d) a polynucleotide of at least 70, at least 80, at least 85, at least 90 percent sequence identity, to a nucleic acid of SEQ ID NO:1 or SEQ ID NO:2;
e) a fragment of at least 16 contiguous nucleotides of a nucleic acid sequence of SEQ ID NO:1 or SEQ ID NO:2; and
f) a complement of the sequence of (a), (b), (c), (d) or (e).

15. The isolated polynucleotide according to claim 14, which
(i) is defined as operably linked to a heterologous promoter; or
(ii) is defined as comprised on a plant transformation vector.

16. A plant transformed, transduced or transfected with a polynucleotide according to claim 1.

17. The plant according to claim 16, wherein said polynucleotide is expressed in a cell of the plant as a double stranded ribonucleotide sequence and uptake of a target pathogen inhibitory amount of said double stranded ribonucleotide sequence and/or of an RNAi inducing compound derived from said double stranded ribonucleotide sequence inhibits the target pathogen from further infections, optionally wherein:
(i) the target pathogen is an aflatoxin-producing fungus belonging to the phylum Ascomycota, in particular the optionally belonging to the order Eurotiales, in particular belonging to the family Trichocomaceae and in particular to a fungus belonging to the genera of Aspergillus, in particular Aspergillus flavus and/or Aspergillus parasiticum and/or Aspergillus fumigatus,
(ii) uptake of the target pathogen inhibitory amount of the double stranded ribonucleotide sequence or fragments thereof stunts the growth of the aflatoxin-producing pathogenic fungi.

18. A method for controlling aflatoxin-production and/or fungal infection comprising providing an agent comprising a first polynucleotide sequence that functions upon uptake by the fungi to inhibit a biological function within said fungi, wherein said polynucleotide sequence exhibits from about 95 to about 100 percent nucleotide sequence identity along at least from about 16 to about 30 contiguous nucleotides to a CSP coding sequence derived from said fungi and is hybridized to a second polynucleotide sequence that is complementary to said first polynucleotide sequence.

19. The method according to claim 18, wherein said CSP coding sequence derived from said fungi is selected from the group consisting of SEQ ID NO:1, SEQ ID NO:2, and a complement or a homolog thereof, optionally said homolog has a sequence identity of at least 60%, optionally of at least 70%, optionally of at least 80%, optionally of at least 85%, optionally of at least 90%, optionally of at least 95%, optionally of at least 96, 97, 98 or 99% to SEQ ID NO:1 or SEQ ID NO:2.

20. A transgenic plant comprising a gene coding an inhibitor against CSP of a target pathogen, wherein said inhibitor inhibits the CSP expression and/or binds to a protein product of a gene coding CSP.

21. A transgenic plant comprising a gene coding an inhibitor against CSP of a target pathogen, wherein said inhibitor inhibits the CSP expression and/or binds to a protein product of a gene coding CSP, wherein the pathogen is a pathogen as defined in claim 2.

22. A transgenic plant comprising a gene coding an inhibitor against CSP of a target pathogen, wherein said inhibitor inhibits the CSP expression and/or binds to a protein product of a gene coding CSP, wherein the inhibitor is a compound as defined in claim 4.

23. An inhibitor against the cysteine-rich secreted protein (CSP) for the use in the treatment of a disease associated with a pathogen expressing CSP and producing aflatoxin.

24. An inhibitor against the cysteine-rich secreted protein (CSP) for the use in the treatment of a disease associated with a pathogen expressing CSP and producing aflatoxin, wherein the pathogen is a pathogen as defined in claim 2.

25. An inhibitor against the cysteine-rich secreted protein (CSP) for the use in the treatment of a disease associated with a pathogen expressing CSP and producing aflatoxin, wherein the inhibitor is a compound as defined in claim 4.

26. A small interfering ribonucleic acid (siRNA) for inhibiting the expression of a cysteine-rich secreted protein (CSP) protein in a target pathogen, wherein the siRNA comprises at least 2 sequences that are complementary to each other and wherein a sense strand comprises a first sequence and an anti-sense strand comprises a second sequence comprising a region of complementarity, which is substantially complementary to at least a part of an mRNA encoding a nucleotide sequence from SEQ ID NO:1.

27. The siRNA according to claim 26, wherein the siRNA contains the sequence of SEQ ID NO:2, or a homolog thereof, optionally said homolog having a sequence identity of at least 60%, optionally of at least 70%, optionally of at least 80%, optionally of at least 85%, optionally of at least 90%, optionally of at least 95%, optionally of at least 96, 97, 98 or 99% to SEQ ID NO: 2.

28. A method of treating a disease associated with a pathogen as defined by claim 2, the method comprising administering an effective amount of an inhibitor to a patient in need thereof wherein the inhibitor is a compound selected from the group consisting of the following (a) to (f):
(a) an RNAi inducing compound that targets a nucleic acid coding CSP or parts thereof;
(b) a nucleic acid construct intracellularly producing an RNAi inducing compound targeted at a nucleic acid coding CSP or parts thereof;
(c) an antisense nucleic acid targeted at the transcript product of a gene coding CSP or parts thereof;
(d) a ribozyme targeted at the transcript product of a gene coding CSP or parts thereof;
(e) a small chemical molecule targeted at the protein product of a gene coding CSP;
(f) a peptide or polypeptide targeted at the protein product of a gene coding CSP.

29. The method according to claim 28, wherein the disease is selected from the group consisting of acute hepatic necrosis, liver damage, liver cirrhosis, liver cancer, mental impairment, abdominal pain, vomiting, convulsions, edema, pulmonary edema, hemorrhaging and disruption of food digestion, absorption or metabolism.


SEQUENCE LISTING (5 sequences)

SEQ ID NO: 1 - DNA, 1017 bp, Aspergillus flavus
atgaagccttctcaactcagcctgctggtcctcctcttccaggcctcctccattcaggccaagacgtacaagaagaatgtcccgacattcgaagttggccctgatgtggtcatcaccgacaacaagattgagtacgatgatcccgactgtgatcccgggtttacatgcagaacaagcaagacctgtgccgcgccgggtaccgttcccaccctcaccggcgacaagaagtacttttcttgctgcttgaagggactgaaccttcttggtagccctgagacagcattcgactgctgtgccgaaggccatgaccttgctggctctggggaagttggctataggtgctgcccaactggacagatctatgacggcctgatctgcaagccggtatgcgcgaatggcaaagttcttgtcgacggcaagtgtgtctgcccgaaagacacagtcgaaggtccggacggggcctgccatgaacaaatctgcacttccgggttaacatctggtaaatgctacaccttcaccgcacccaatggcaacaccctaggctccggcgccgacggaatctactacgccaaacccgacgacatgaacttccactatggtaaattccagctttgtctggacgagaagtgcgagggcaacctccccatcaacccccaggacggagtgtacattagagacctgtacggcgacgtcaagaccggcgcgaacaagggccagtggctcaacaatgcaaaggatggcgcccatatcggcaagaccaaggactttgccgcagccggcaaattctctttgagcaagtggccatgcggaaagtactgtcttggaggcgttgagtggggtgtcggtcccgcttgtccttctctgacacctgcgattaccttcttctcacaggatccgcagatgtgtactgcctttgatctcactgagatcccttgtgatattaaggcgcctgccaataactgtatttggaagagtgggaagaaccagtgctgtggtaaggttgattgcggtctgtga

SEQ ID NO: 2 - DNA, 27 bp, Artificial Sequence
  siRNA (SEQ ID NO.2) derived from SEQ ID NO.1
gacagatctatgacggcctgatctgca

SEQ ID NO: 3 - protein, 338 aa, Aspergillus flavus
MKPSQLSLLVLLFQASSIQAKTYKKNVPTFEVGPDVVITDNKIEYDDPDCDPGFTCRTSKTCAAPGTVPTLTGDKKYFSCCLKGLNLLGSPETAFDCCAEGHDLAGSGEVGYRCCPTGQIYDGLICKPVCANGKVLVDGKCVCPKDTVEGPDGACHEQICTSGLTSGKCYTFTAPNGNTLGSGADGIYYAKPDDMNFHYGKFQLCLDEKCEGNLPINPQDGVYIRDLYGDVKTGANKGQWLNNAKDGAHIGKTKDFAAAGKFSLSKWPCGKYCLGGVEWGVGPACPSLTPAITFFSQDPQMCTAFDLTEIPCDIKAPANNCIWKSGKNQCCGKVDCGL

SEQ ID NO: 4 - protein, 138 aa, Artificial Sequence
  Antibody mAbAP10 heavy chain variable regions
MEWSWIFLFLLSGTAGVHSMADVQLVESGGGLVQPGGSRKLSCAASGFTFSSFGMHWVRQAPEKGLEWVAYINGGSGTIYYADTVKGRFTISRDNPRNTLFLQMETTSLRSEDTAMYYCARGGDDPFAYWGQGTLVTV

SEQ ID NO: 5 - protein, 105 aa, Artificial Sequence
  Antibody mAbAP10 light chain variable regions
IQMTQSPSSLSAYLGDKVTITCMASQDINNHIAWYQLKPGKGPRLLIHYTSTLHPGIPSRFSGSGSGRDYSFSISDLEPEDIATYYCLQYDNLRTFGGGTKLEIK